SEQUENCE LISTING (27 sequences)

SEQ ID NO: 1 - DNA, 18 bp, Artificial
  A PCR primer
tagacgggctcacatcac

SEQ ID NO: 2 - DNA, 24 bp, Artificial
  A PCR primer
ggggtatctaatcccagtttgggt

SEQ ID NO: 3 - DNA, 14 bp, Artificial
  A PCR primer
cccataaacaaata

SEQ ID NO: 4 - DNA, 30 bp, Artificial
  A PCR primer
taggtcaaggtgtagcccatgaggtggcaa

SEQ ID NO: 5 - DNA, 21 bp, Artificial
  A PCR primer
gtaaggtggagtgggtttggg

SEQ ID NO: 6 - DNA, 18 bp, Artificial
  A PCR primer
acgcatttatatagagga

SEQ ID NO: 7 - DNA, 20 bp, Artificial
  A PCR primer
aggacaagagaaataaggcc

SEQ ID NO: 8 - DNA, 20 bp, Artificial
  A PCR primer
acgttggggcctttgcgtag

SEQ ID NO: 9 - DNA, 15 bp, Artificial
  A PCR primer
aagaagaggaattga

SEQ ID NO: 10 - DNA, 20 bp, Artificial
  A PCR primer
aggacaagagaaataaggcc

SEQ ID NO: 11 - DNA, 20 bp, Artificial
  A PCR primer
acgttggggcctttgcgtag

SEQ ID NO: 12 - DNA, 18 bp, Artificial
  A PCR primer
tgcgattagaatgggtac

SEQ ID NO: 13 - DNA, 19 bp, Artificial
  A PCR primer
ctcatgcccccatgtctaa

SEQ ID NO: 14 - DNA, 22 bp, Artificial
  A PCR primer
ttacttttatttggagttgcac

SEQ ID NO: 15 - DNA, 13 bp, Artificial
  A PCR primer
tttggagttgcac

SEQ ID NO: 16 - DNA, 22 bp, Artificial
  A PCR primer
cctcacaggtttctactccaaa

SEQ ID NO: 17 - DNA, 25 bp, Artificial
  A PCR primer
aagtcctaggaaagtgacagcgagg

SEQ ID NO: 18 - DNA, 15 bp, Artificial
  A PCR primer
aggtttctactccaa

SEQ ID NO: 19 - DNA, 22 bp, Artificial
  A PCR primer
cagcttcctacactattaaagt

SEQ ID NO: 20 - DNA, 23 bp, Artificial
  A PCR primer
gtttttttaatttatttaggggg

SEQ ID NO: 21 - DNA, 18 bp, Artificial
  A PCR primer
gatactcctcaatagcca

SEQ ID NO: 22 - DNA, 22 bp, Artificial
  A PCR primer
cagcttcctacactattaaagt

SEQ ID NO: 23 - DNA, 23 bp, Artificial
  A PCR primer
gtttttttaatttatttaggggg

SEQ ID NO: 24 - DNA, 14 bp, Artificial
  A PCR primer
gcgccaaggagtga

SEQ ID NO: 25 - DNA, 22 bp, Artificial
  A PCR primer
cagcttcctacactattaaagt

SEQ ID NO: 26 - DNA, 23 bp, Artificial
  A PCR primer
gtttttttaatttatttaggggg

SEQ ID NO: 27 - DNA, 20 bp, Artificial
  A PCR primer
tttctgagtagagaaatgat

(19) United States
(12) Patent Application Publication
(10) Pub. No.: US 2013/0136726 A1
(43) Pub. Date: May 30, 2013

(54) METHOD FOR DETECTION OF PREDISPOSITION TO ATHEROSCLEROSIS, CORONARY HEART DISEASE AND RELATED CONDITIONS

(71) Applicants: Igor Sobenin, Krasnogorsk (RU); Alexander Orekhov, Moscow (RU); Jukka Salonen, Helsinki (FI)

(72) Inventors: Igor Sobenin, Krasnogorsk (RU); Alexander Orekhov, Moscow (RU); Jukka Salonen, Helsinki (FI)

(73) Assignee: MAS-METABOLIC ANALYTICAL SERVICES OY, Helsinki (FI)

(21) Appl. No.: 13/698,535

(22) PCT Filed: May 19, 2011
(86) PCT No.: PCT/FI2011/050459

§ 371 Date: Feb. 1, 2013

(30) Foreign Application Priority Data
May 19, 2010 (FI) 20100211

(51) Int. Cl. C12Q 1/68; A61K 48/00; A61K 31/7088; A61K 38/44

(52) U.S. Cl. 424/94.4; 435/6.11; 514/44 R; 514/44 A

(57) ABSTRACT

Heteroplasmy mitochondrial DNA (mtDNA) markers and haplotypes of susceptibility or predisposition to atherosclerosis, coronary heart disease (CHD) and subdiagnosis of atherosclerosis and CHD and related medical conditions are disclosed. The biomarkers may be selected from the following heteroplasmy makers: 652lns/del G; A1555G; C3256T; T3336C; G12315A; G13513A; G14459A; G14846A; G15059A. Methods and kits for diagnosis, subdiagnosis, and prediction of clinical course and efficacy of treatments for CHD, atherosclerosis and related phenotypes using heteroplasmy in the risk genes and loci and other related biomarkers are thus provided. Novel methods for prevention and treatment of atherosclerosis, CHD and related conditions based on the disclosed CHD genes, loci, polypeptides and related pathways are also provided.

### BACKGROUND OF THE INVENTION

The energy metabolism is critically important for life and its defects cause a number of severe metabolic disorders and disease conditions in humans. The energy metabolism in mitochondria is an important source of reactive oxygen species (ROS), free radicals and oxidative stress, which on one hand regulate the metabolome widely and on the other contribute to the initiation and progress of a number of diseases such as atherosclerosis and its consequences. ROS are also believed to induce mitochondrial damage. At higher concentrations, ROS can cause cell injury and death. ROS are also involved in hypertension and obesity and type 2 diabetes.

In human pathology, several diseases have been associated with somatic mutations in the mitochondrial genome. These mitochondrial mutations arise during ontogenesis and are associated with pathologies such as coronary vessel stenosis, some forms of diabetes and deafness, myocardial infarction, cardiomyopathy, and atherosclerosis [1-17].

The mammalian mitochondrial genome (mtDNA) is a small double-stranded DNA molecule that is exclusively transmitted down the maternal line. The human mitochondrial DNA is a ringed two-chain molecule consisting of 16,569 nucleotide pairs that encode 37 genes. Twenty-two genes encode transport RNAs (tRNAs), 2 genes encode ribosomal RNAs (rRNAs), and 13 genes encode subunits of the respiration chain complex such as cytochrome B, ATPase, cytochrome-C-oxidase, and NADH-dehydrogenase. A mitochondrion usually contains multiple copies of its genome. The maternally inherited mitochondrial genome is characteristically unstable; thus, the occurrence of somatic mutations during the life of an individual is common. The penetrance and expressivity of such mutations vary widely between families, and between relatives (in the maternal line) within a family. Although many factors influence penetrance and expressivity, two main factors are genotype and the level of heteroplasmy (mixture of mutant and normal DNA molecules) [18].

Heteroplasmy is defined as the presence of a mixture of more than one type of an organellar genome (mitochondrial DNA (mtDNA) or plastid DNA) within a cell or individual. Pathogenic mtDNA mutations are usually heteroplasmic, with a mixture of mutant and wild-type mtDNA within the same organism. A woman harbouring one of these mutations transmits a variable amount of mutant mtDNA to each offspring. Heteroplasmy, the presence of more than one type of mtDNA within cells, is common in animals and has been associated with aging and disease in humans. Mitochondrial DNA is present in hundreds to thousands of copies per cell and also has a very high mutation rate. New mtDNA mutations arise in cells, coexist with wild-type mtDNAs (heteroplasmy), and segregate randomly during cell division. The vast majority of deleterious mtDNA point mutations are heteroplasmic and their mutant load can vary significantly among different tissues, even in the same subject. Heteroplasmic mtDNA defects are considered an important cause of human disease with clinical features that primarily involve nondividing (postmitotic) tissues. The proportion of mutant out of total mtDNA in a cell, called the heteroplasmy level, is an important factor in determining the amount of mitochondrial dysfunction and therefore the disease severity.

Sazonova and coworkers have developed a mutant allele quantitative assay to study differences in tissue-specific mitochondrial mutations between lipofibrous atherosclerotic plaques and normal arterial tissue. The level of heteroplasmy of 40 mitochondrial mutations previously identified in several pathological conditions was assessed in human aortic intimal tissue. These were located in MT-RNR1, MT-TL1, MT-TL2, MT-TW, MT-TN, MT-TC, MT-TK, MT-TE, MT-CO1, MT-CO3, MT-ND1, MT-ND2, MT-ND5, MT-ND6, MT-ATP-6 and MT-CYB genes. Eleven mitochondrial mutations in 8 genes (MT-RNR1, MT-TL1, MT-TL2, MT-ND1, MT-ND2, MT-ND5, MT-ND6 and MT-CYB, which encode 12S rRNA, tRNA-Leu, cytochrome B, and subunits 1, 2, 5, and 6 NADH dehydrogenase) had higher levels of heteroplasmy in atherosclerotic plaques as compared with normal intima [19]. Sazonova et al. did not study associations of somatic mtDMA mutations in other cells such as leukocytes.

Takagi et al. observed an association between the presence of a 5178C>A polymorphism in mtDNA with the prevalence of myocardial infarction in Japanese individuals [20]. This observation has been repeated by others.

The thickness of the intima-media layer of carotid arteries (cIMT), determined by high-resolution ultrasonography is considered to be generally accepted non-invasive marker of subclinical atherosclerosis used in clinical and epidemiological studies to assess the impact of traditional and new factors of cardiovascular risk in the development of atherosclerosis [21,22].

Since there is a correlation of cIMT with the degree of coronary atherosclerosis [21,22], and cIMT has predictive significance with regard to the clinical manifestations of atherosclerosis [21-23], it is proposed as a surrogate marker of systemic (including coronary) atherosclerosis. The classical factors of cardiovascular risk are poorly associated with cIMT [22,24], which suggests the presence of other factors determining the risk of atherosclerosis such as hereditary factors.

### SUMMARY OF THE INVENTION

We believe that both qualitative (presence or absence of a mutation) and quantitative (presence of heteroplasmy OR heteroplasmy percentage) estimations of mutant alleles in the mitochondrial genome are necessary for studying the association between mitochondrial mutations and human diseases.

White blood cells, especially blood-derived monocytes-macrophages play a special role in atherogenesis. They migrate in the subendothelial layer in arteries and participate in the processes of inflammation and atherosclerotic plaque formation. Also other leukocytes have important roles in atherogenesis. The present invention is based on the hypothesis that the higher the level of mtDNA heteroplasmy in circulating monocytes, the higher the likelihood that the defective monocytes enter into the arterial intimal layer. If monocyte cell function is inhibited due to the presence of mutations in coding region of mtDNA, this may lead to local oxidative stress and other pathologic events, which could promote atherosclerosis formation. The same concerns also other white blood cells such as the neutrophils. We therefore assume that mtDNA heteroplasmy and other biomarkers of defective mitochondrial function in blood leukocytes are biomarkers of atherogenesis, atherosclerosis and consequent clinical manifestations such as coronary heart disease, cerebrovascular disease, intermittent claudication and congestive heart failure.

As free radicals and lipid peroxidation have been previously shown to be relevant in the etiology of atherosclerosis and CHD [25], among genetic factors, we hypothesized that leukocyte mitochondrial mutations would have a role in atherosclerosis and CHD.

Early detection and treatment of patients with high risk for atherosclerosis is an urgent medical, public health and social problem, the solution of which should lead to lower cardiovascular morbidity and mortality. For this task, the identification of markers of subclinical atherosclerosis is important.

This invention describes novel diagnostic biomarkers for atherosclerosis and related cardiovascular diseases such as coronary heart disease (CHD), cerebrovascular disease, intermittent claudication, congestive heart failure and other manifestations of arteriosclerosis, hypertension, obesity and type 2 diabetes. The present invention provides novel genes, loci and individual biomarkers associated with these conditions. The invention further relates to physiological and biochemical routes and pathways related to these genes, as well as gene and other therapies modifying the genes or their products.

The detection of the biomarkers of this invention provides novel methods and systems for risk assessment and diagnosis of atherosclerosis, which will also improve risk assessment, diagnosis and prognosis of atherosclerosis related conditions comprising coronary complications, coronary artery disease, myocardial infarction, angina pectoris, cerebrovascular stroke, claudication and congestive heart failure.

The present invention particularly provides a method for risk assessment, diagnosis, subdiagnosis or prognosis of atherosclerosis, coronary heart disease (CHD) or an atherosclerosis or CHD related condition in a mammalian subject comprising:
a) providing a biological sample selected from the group consisting of a blood, saliva, urine, mucosal, and hair shaft sample taken from the subject;
b) detecting one or more CHD and/or atherosclerosis or related phenotype associated biomarkers in said sample, wherein the biomarkers are related to one or more genes selected from the group consisting of MT-ND5, MT-RNR1, MT-TL1, MT-TL2, MT-ND1, MT-ND6 and MT-CYB genes, which encode subunit 5 of NADH dehydrogenase, 12S rRNA, tRNA-Leu 1, tRNA-Leu 2, subunits 1 and 6 of NADH dehydrogenase, and cytochrome B, respectively, or said biomarkers are related to one or more polypeptides encoded by said genes, and;
c) comparing the biomarker data from the subject to biomarker data from healthy and diseased people to make risk assessment, diagnosis, subdiagnosis or prognosis of atherosclerosis, CHD or a CHD related condition.

Another application of the current invention is its use to predict an individual's response to a particular atherosclerosis preventing or treating or anti-coronary or antihypertensive or anti-diabetic or weight-reduction method of therapy. It is a well-known phenomenon that in general, patients do not respond equally to the same drug, food or other therapy. Much of the differences in the response to a given therapy are thought to be based on genetic and protein differences among individuals in certain genes and their corresponding pathways. Our invention defines the genes and loci associated with a response to known method(s) of therapy in atherosclerosis, CHD and related conditions. Therefore, genes and mutations which are the subject of current invention may be used in pharmacogenetic and nutrigenetic diagnostics to predict a response to a method of therapy and guide choice of method(s) of therapy for treating, preventing or ameliorating the symptoms, severity or progression of atherosclerosis and CHD or a CHD related condition in a given individual (“personalized nutrition”, “personalized medicine”, “personalized prevention”).

Still another object of the invention is to provide a method for prediction of clinical course, and efficacy and safety of therapeutic method(s) with current anti-atherosclerotic, anticoronary, antihypertensive, glucose lowering and weight-reduction drugs, foods and other therapies for CHD using the levels of heteroplasmies in the loci associated with such response.

Another object of the invention is providing novel pathways to elucidate the presently unknown modes of action of known anti-atherosclerosis, anti-coronary, antihypertensive, glucose lowering and weight-reduction medicines, foods and diets. A major object of the invention are gene networks influencing individual's response to a method of therapy. Such gene networks can be used for other methods of the invention comprising diagnostic methods for prediction of the response to a particular medicine or food, the efficacy and safety of a particular method of therapy described herein and the treatment methods described herein.

Kits are also provided for the selection, prognosis and monitoring of the method of therapy for atherosclerosis, hypertension, obesity and type 2 diabetes. Better means for identifying those individuals who will benefit more from the selected method of therapy for atherosclerosis, CHD, hypertension, obesity and type 2 diabetes due to the better response and fewer adverse effects should lead to better preventive and treatment regimens. Pharmacogenetic information may be used to assist physician in choosing method of therapy for the particular patient (“personalized medicine”).

In summary, the invention helps meet unmet medical needs and promotes public health in at least two major ways: 1) it provides novel means to predict individual's predisposition to atherosclerosis and related conditions and response and evaluate safety and efficiency of a selected method of therapy with known atherosclerosis preventing or treating or anti-coronary, antihypertensive, anti-obesity or antidiabetic medicine, food or other therapy, as well as select the significant suitable alternative method of anti-atherosclerosis or anti-coronary or related therapy for the individual (“personalized medicine”, “personalized nutrition”) and 2) it provides therapeutic targets that can be used further to screen and develop small molecule drugs, biologicals, gene therapies, functional foods and other therapeutic agents and therapies that can be used alone or in combination with the known anti-atherosclerosis and anti-coronary and related therapies to treat, prevent or ameliorate the symptoms, severity or progression of atherosclerosis and CHD or a CHD related condition in a given individual.

Accordingly in a first aspect, the present invention provides methods and kits for diagnosing a susceptibility to develop atherosclerosis or related conditions in an individual. The methods comprise the steps of: (i) obtaining a biological sample from the individual, and (ii) detecting in the biological sample the presence of one or more atherosclerosis and/or CHD associated biomarkers. These biomarkers may be qualitative or quantitative measures of heteroplasmy selected from Table 3 or the Figures of the invention or other biomarkers of the loci that they are associated with such as expressed RNA or protein or metabolites of the protein. The presence or absence or high or low amount of atherosclerosis associated biomarkers in subject's sample is indicative of a susceptibility to atherosclerosis or related condition. The kits provided for diagnosing a susceptibility to atherosclerosis or related condition in an individual comprise wholly or in part protocol and reagents for detecting one or more biomarkers and interpretation software for data analysis and risk assessment. In one embodiment of this invention alleles in loci being in linkage disequilibrium with one or more mutations of this invention are used in methods and kits for diagnosing a susceptibility to atherosclerosis. In other embodiment metabolites, expressed RNA molecules or expressed polypeptides, which are associated with one or more Heteroplasmy markers of this invention are used in disclosed methods and kits.

In one typical embodiment, the biomarker information obtained from the methods diagnosing a susceptibility of an individual to atherosclerosis or related condition are combined with other information concerning the individual, e.g. results from blood measurements, clinical examination, questionnaires and/or interviews. The present invention suggests novel measurements for highly effective identification of patients predisposed to atherosclerosis.

In one embodiment, the methods and kits of the invention are used in early diagnosis of atherosclerosis, CHD, hypertension, obesity and type 2 diabetes at or before onset, thus reducing or minimizing the debilitating effects of these conditions, in “premorbial prevention”. In a preferred embodiment the methods and kits are applied in individuals who are free of clinical symptoms and signs of atherosclerosis and/or CHD, but have family history of atherosclerosis, CHD, hypertension, obesity and/or type 2 diabetes or in those who have multiple risk factors for these conditions.

In a second aspect, the present invention provides methods and kits for molecular diagnosis i.e. determining a molecular subtype of atherosclerosis, CHD, hypertension, obesity and type 2 diabetes in an individual. In one preferred embodiment, molecular subtype of atherosclerosis in an individual is determined to provide information of the molecular etiology of atherosclerosis and related conditions. When the molecular etiology is known, better diagnosis and prognosis can be made and efficient and safe therapy for treating atherosclerosis or related condition in an individual can be selected on the basis of this subtype information. For example, the medicine, food, gene therapy or other therapy that is likely to be effective, can be selected without trial and error. As another example, an individual with a lot or little of heteroplasmy in the rRNA 12S, tRNA-Leu, cytochrome B or NADH dehydrogenase or under-expressed or defective or over-expressed or overactive cytochrome B or NADH dehydrogenase may benefit from therapies affecting this enzyme. The therapy may be gene therapy, small-molecule drug, a biological preparation, or a functional food.

In another embodiment, biomarker information obtained from methods and kits for determining molecular subtype of atherosclerosis or related condition in an individual is for monitoring the effectiveness of atherosclerosis treatment. In one embodiment, methods and kits for determining molecular subtype of atherosclerosis are used to select human subjects for clinical trials testing efficacy of therapies for atherosclerosis or related condition. The kits provided for diagnosing a molecular subtype of atherosclerosis in an individual comprise wholly or in part protocol and reagents for detecting one or more biomarkers and interpretation software for data analysis and atherosclerosis molecular subtype assessment.

As mtDNA heteroplasmies are to an extent tissue-specific, this invention also concerns tissue-specific heteroplasmies. For example, mtDNA heteroplasmy may be assessed in the myocardial and arterial tissue which are relevant with regard to coronary heart disease and atherosclerosis and its consequences, adipose, muscle tissues and gastric which are relevant for obesity and type 2 diabetes, and pancreatic tissue with is relevant to type 2 diabetes. In one embodiment, the biomarkers of this invention are heteroplasmies of mtDNA in myocardium, arterial wall, adipose tissue, muscle tissue, pancreatic or gastric tissue. In the empirical examples, we assessed mtDNA heteroplasmies in arterial tissue of necropsy samples and blood leukocytes. However, myocardium- and artery-specific levels of heteroplasmies can also be determined in living individuals. At the moment, mitochondrial DNA samples can be obtained by microbiopsy. These can also be taken from adipose, muscle, pancreatic or gastric tissue. In the future, mtDNA heteroplasmies may also be determined by non-invasive methods such as nuclear magnetic resonance (NMR) spectroscopy or other developed molecular imaging techniques.

In our examples, we used blood leukocyte mtDNA heteroplasties to reflect the genomic situation in the whole body. Also other tissues can be used for this purpose, for example urine, saliva and oral or other mucosa.

### BRIEF DESCRIPTION OF THE DRAWINGS

FIG. 1. An association of mutational burden with the extent of carotid atherosclerosis. Mutational burden was estimated as the sum of ranked values (quartile numbers) of percent of heteroplasmy for each mutation according to the sign of beta coefficients obtained from linear regression model. Boxplots show median and interquartile ranges of mutational burden, open circles define outliners. NA, non-atherosclerotic patients (evidently normal thickness of intima-media complex); DIT, abnormal diffuse intimal thickening; AP, abnormal diffuse intimal thickening along with atherosclerotic plaque.

FIG. 2. An association of mutational excess with the extent of carotid atherosclerosis. Mutational excess was estimated as the sum of ranked values (quartile numbers) of percent of heteroplasmy for 4 mutations, which were associated with the degree of atherosclerosis in linear regression model with p<0.001 (13513 G→A, 3256 C→T, 15059 G→A, and 12315 G→A), according to the sign of beta coefficients obtained from linear regression model. Boxplots show median and interquartile ranges of mutational burden, open circles define outliners. NA, non-atherosclerotic patients (evidently normal thickness of intima-media complex); DIT, abnormal diffuse intimal thickening; AP, abnormal diffuse intimal thickening along with atherosclerotic plaque.

FIG. 3. Receiver operating characteristics curve for mutational burden as the marker of the presence of atherosclerotic plaque. This curve demonstrates sensitivity and specificity of the estimate of mutational burden; positive real state is the presence of atherosclerotic plaque. Area under curve (AUC) is 0.975 (95% CI 0.954-0.966, p<0.001).

FIG. 4. Receiver operating characteristics curve for mutational burden as the marker of the presence of subclinical atherosclerosis. This curve demonstrates sensitivity and specificity of the estimate of mutational burden; positive real state is the presence of subclinical disease (abnormal diffuse intimal thickening regardless to the presence of absence of plaque in the basin of carotid arteries). Area under curve (AUC) is 0.986 (95% CI 0.973-0.999, p<0.001).

FIG. 5. Receiver operating characteristics curve for mutational excess as a marker of the presence of atherosclerotic plaque. This curve demonstrates sensitivity and specificity of the estimate of mutational excess; positive real state is the presence of atherosclerotic plaque. Area under curve (AUC) is 0.997 (95% CI 0.993-1.001, p<0.001)

FIG. 6. Receiver operating characteristics curve for mutational excess as the marker of the presence of subclinical atherosclerosis. This curve demonstrates sensitivity and specificity of the estimate of mutational excess; positive real state is the presence of subclinical disease (abnormal diffuse intimal thickening regardless to the presence of absence of plaque in the basin of carotid arteries). Area under curve (AUC) is 0.988 (95% CI 0.977-0.999, p<0.001)

FIG. 7. Receiver operating characteristics curve for 13513 G→A mutation as the marker of the absence of subclinical atherosclerosis. This curve demonstrates sensitivity and specificity of the level of heteroplasmy of 13513 G→A mutation; positive real state is evidently normal thickness of intima-media complex, because this marker is negatively associated with the predisposition to atherosclerosis. Area under curve (AUC) is 0.920 (95% CI 0.880-0.961, p<0.001).

FIG. 8. Receiver operating characteristics curve for 3256 C→T mutation as the marker of the presence of subclinical atherosclerosis. This curve demonstrates sensitivity and specificity of the level of heteroplasmy of 3256 C→T mutation; positive real state is the presence of subclinical disease (abnormal diffuse intimal thickening regardless to the presence of absence of plaque in the basin of carotid arteries). Area under curve (AUC) is 0.819 (95% CI 0.754-0.885, p<0.001).

FIG. 9. Receiver operating characteristics curve for 15059 G→A mutation as the marker of the presence of subclinical atherosclerosis. This curve demonstrates sensitivity and specificity of the level of heteroplasmy of 15059 G→A mutation; positive real state is the presence of subclinical disease (abnormal diffuse intimal thickening regardless to the presence of absence of plaque in the basin of carotid arteries). Area under curve (AUC) is 0.983 (95% CI 0.963-1.003, p<0.001).

FIG. 10. Receiver operating characteristics curve for 12315 G→A mutation as the marker of the presence of subclinical atherosclerosis. This curve demonstrates sensitivity and specificity of the level of heteroplasmy of 12315 G→A mutation; positive real state is the presence of subclinical disease (abnormal diffuse intimal thickening regardless to the presence of absence of plaque in the basin of carotid arteries). Area under curve (AUC) is 0.817 (95% CI 0.749-0.885, p<0.001).

FIG. 11. Receiver operating characteristics curve for 14459 G→A mutation as the marker of the presence of subclinical atherosclerosis. This curve demonstrates sensitivity and specificity of the level of heteroplasmy of 14459 G→A mutation; positive real state is the presence of subclinical disease (abnormal diffuse intimal thickening regardless to the presence of absence of plaque in the basin of carotid arteries). Area under curve (AUC) is 0.705 (95% CI 0.622-0.788, p<0.001).

FIG. 12. Receiver operating characteristics curve for 3336 T→C mutation as the marker of the presence of subclinical atherosclerosis. This curve demonstrates sensitivity and specificity of the level of heteroplasmy of 3336 T→C mutation; positive real state is the presence of subclinical disease (abnormal diffuse intimal thickening regardless to the presence of absence of plaque in the basin of carotid arteries). Area under curve (AUC) is 0.877 (95% CI 0.822-0.932, p<0.001).

FIG. 13. Receiver operating characteristics curve for 1555 A→G mutation as the marker of the absence of subclinical atherosclerosis. This curve demonstrates sensitivity and specificity of the level of heteroplasmy of 1555 A→G mutation; positive real state is evidently normal thickness of intima-media complex, because this marker is negatively associated with the predisposition to atherosclerosis. Area under curve (AUC) is 0.715 (95% CI 0.617-0.813, p<0.001).

FIG. 14. Receiver operating characteristics curve for 14846 G→A mutation as the marker of the presence of subclinical atherosclerosis. This curve demonstrates sensitivity and specificity of the level of heteroplasmy of 14846 G→A mutation; positive real state is the presence of subclinical disease (abnormal diffuse intimal thickening regardless to the presence of absence of plaque in the basin of carotid arteries). Area under curve (AUC) is 0.732 (95% CI 0.655-0.808, p<0.001).

FIG. 15. Receiver operating characteristics curve for 652 del G mutation as the marker of the presence of atherosclerotic plaque. This curve demonstrates sensitivity and specificity of the level of heteroplasmy of 652 del G mutation; positive real state is the presence of atherosclerotic plaque. Area under curve (AUC) is 0.581 (95% CI 0.484-0.679, p=0.095).

FIG. 16. Receiver operating characteristics curve for 652 ins G mutation as the marker of the absence of atherosclerotic plaque. This curve demonstrates sensitivity and specificity of the level of heteroplasmy of 652 ins G mutation; positive real state is the absence of atherosclerotic plaque, because this marker is negatively associated with the predisposition to atherosclerosis. Area under curve (AUC) is 0.698 (95% CI 0.615-0.781, p<0.001).

### DETAILED DESCRIPTION OF THE INVENTION

The present invention relates to previously unknown associations between atherosclerosis or related condition and various biomarkers. These novel biomarkers provide basis for novel methods and kits for risk assessment and diagnosis of atherosclerosis and atherosclerosis related conditions.

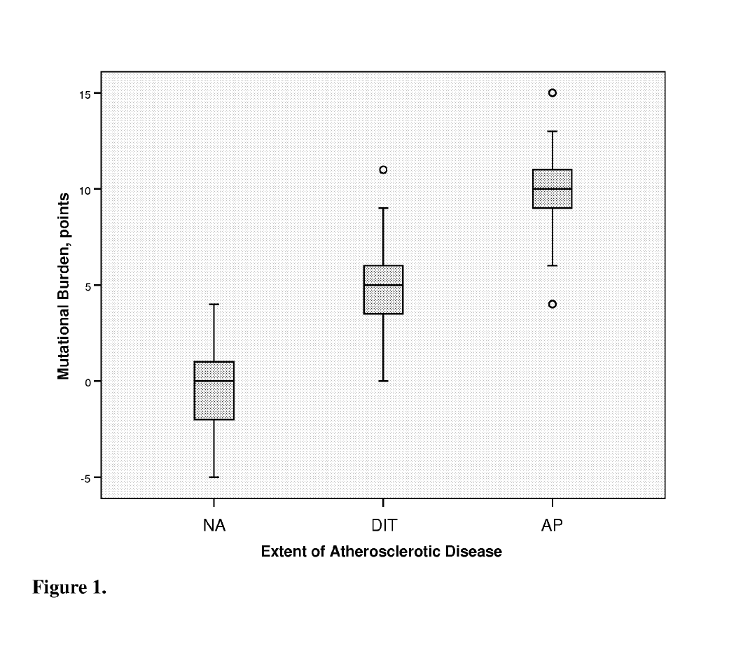

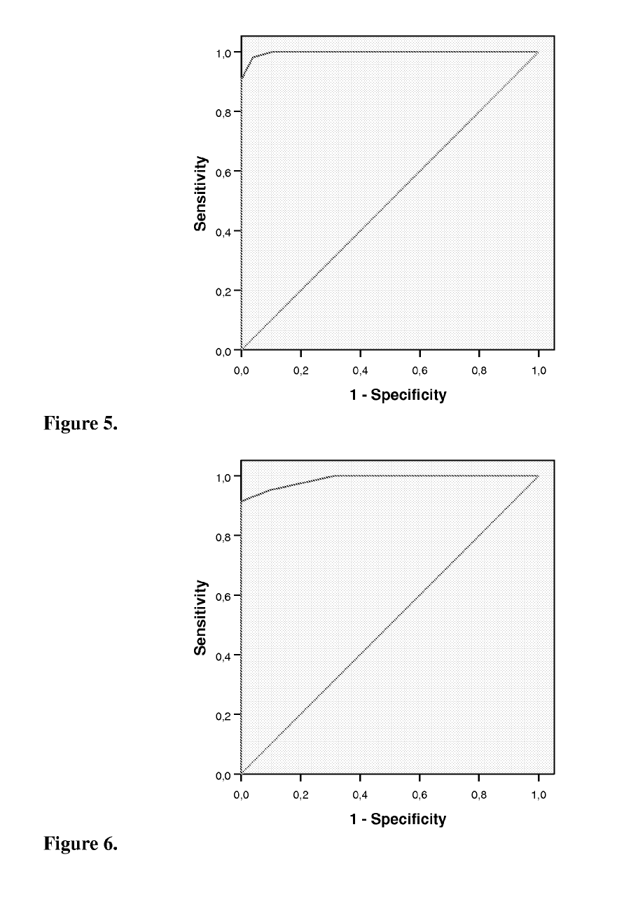

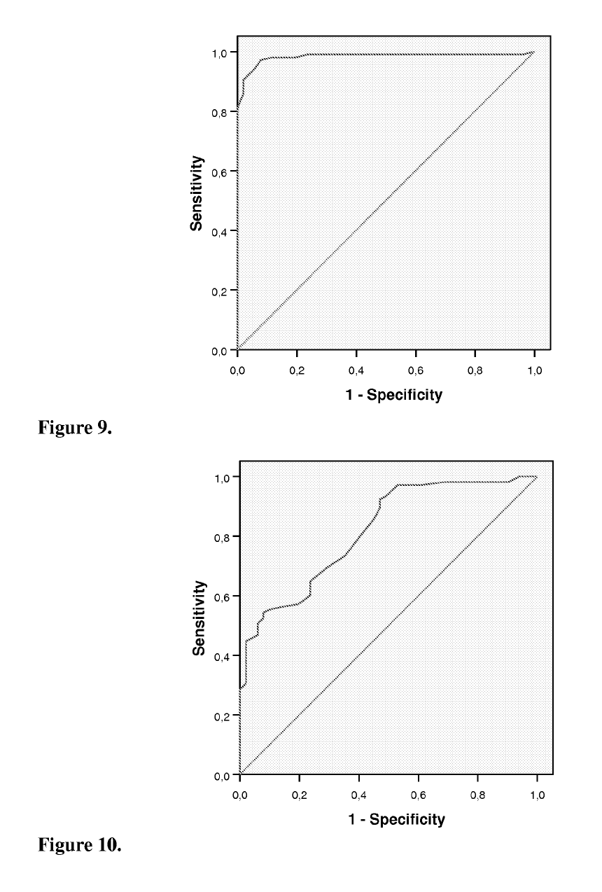

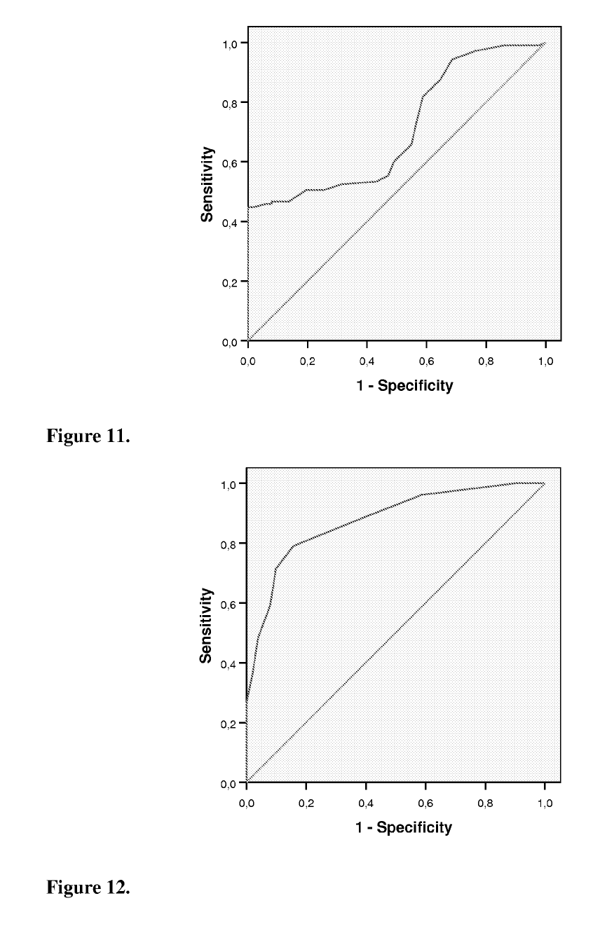

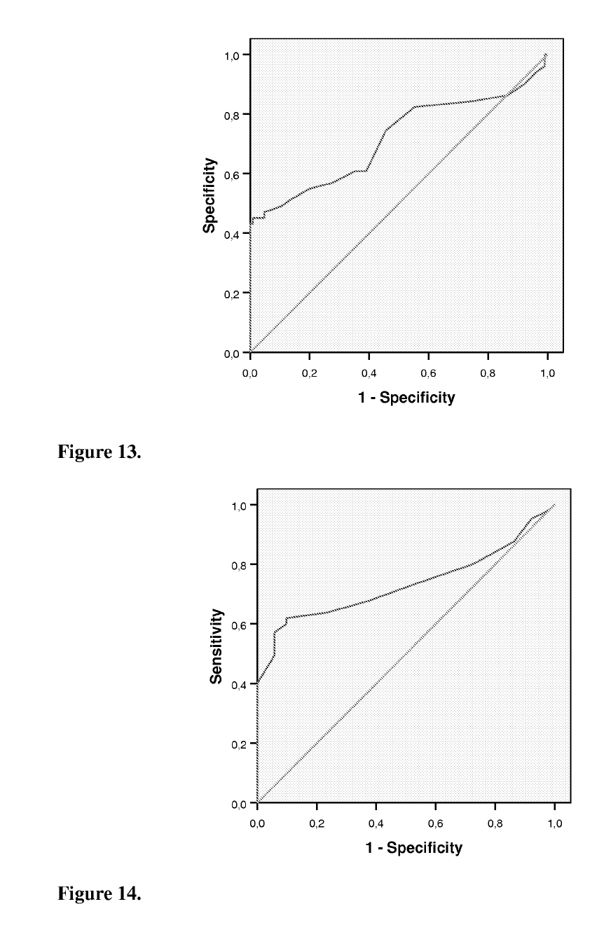

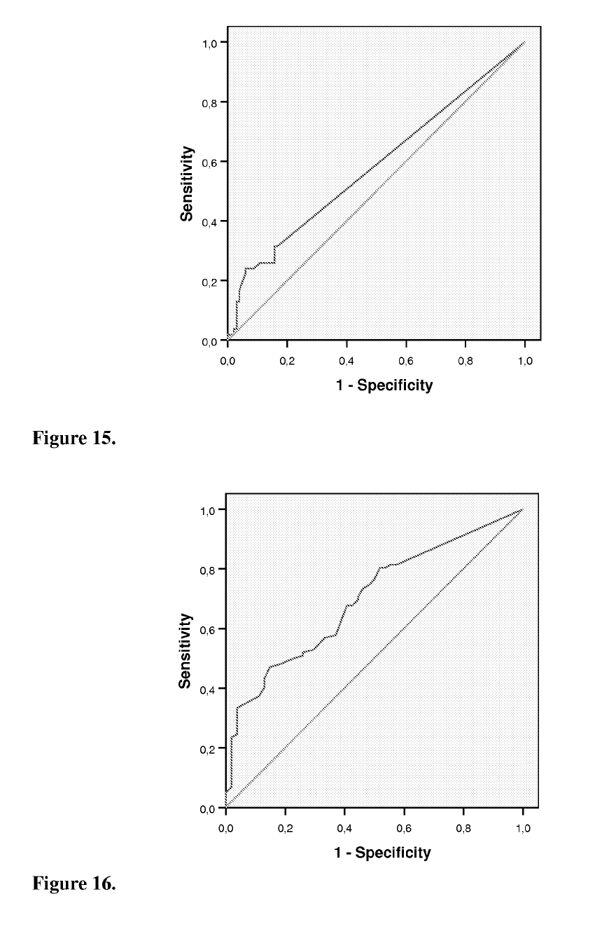

A “biomarker” in the context of the present invention refers to a mutation or degree of heteroplasmy in loci disclosed in Table 3 or FIGS. 1-16 or to a polymorphism, mutation or heteroplasmy which is in linkage disequilibrium with one or more disclosed biomarkers, or to an organic biomolecule which is related to a biomarker set forth in Table 3 or FIGS. 1-16 and which is differentially present in samples taken from subjects (patients) being atherosclerotic or with related condition compared to comparable samples taken from subjects who are non-atherosclerotic. An “organic biomolecule” refers to an organic molecule of biological origin comprising steroids, amino acids, nucleotides, sugars, polypeptides, polynucleotides, complex carbohydrates and lipids. A biomarker is differentially present between two samples if the amount, structure, function or biological activity of the biomarker in one sample differs in a statistically significant way from the amount, structure, function or biological activity of the biomarker in the other sample.

A “haplotype,” as described herein, refers to a combination of genetic markers (“alleles”). A haplotype can comprise two or more alleles and the length of a genome region comprising a haplotype may vary from few hundred bases up to hundreds of kilobases. As it is recognized by those skilled in the art the same haplotype can be described differently by determining the haplotype defining alleles from different nucleic acid strands. E.g., the haplotype TAA defined by the markers C3256T, G12315A and G15059A of this invention. The haplotypes described herein are differentially present in individuals with atherosclerosis than in individuals without atherosclerosis. Therefore, these haplotypes have diagnostic value for risk assessment, diagnosis and prognosis of atherosclerosis in an individual. Detection of haplotypes can be accomplished by methods known in the art used for detecting nucleotides at polymorphic sites. Haplotypes found more frequently in atherosclerotic individuals (risk increasing haplotypes) as well as haplotypes found more frequently in non-atherosclerotic individuals (risk reducing haplotypes) have predictive value for predicting susceptibility to atherosclerosis in an individual.

A nucleotide position in DNA at which more than one sequence is possible in a population, is referred to herein as a “polymorphic site” or “polymorphism”. Where a polymorphic site is a single nucleotide in length, the site is referred to as a SNP. For example, if at a particular chromosomal location, one member of a population has an adenine and another member of the population has a thymine at the same position, then this position is a polymorphic site, and, more specifically, the polymorphic site is a SNP. Polymorphic sites may be several nucleotides in length due to insertions, deletions, conversions or translocations. Each version of the sequence with respect to the polymorphic site is referred to herein as an “allele” of the polymorphic site. Thus, in the previous example, the SNP allows for both an adenine allele and a thymine allele.

Typically, a reference nucleotide sequence is referred to for a particular gene e.g. in NCBI databases (www.ncbi.nlm.nih.gov). Alleles that differ from the reference are referred to as “variant” alleles. The polypeptide encoded by the reference nucleotide sequence is the “reference” polypeptide with a particular reference amino acid sequence, and polypeptides encoded by variant alleles are referred to as “variant” polypeptides with variant amino acid sequences. Nucleotide sequence variants can result in changes affecting properties of a polypeptide. These sequence differences, when compared to a reference nucleotide sequence, include insertions, deletions, conversions and substitutions: e.g. an insertion, a deletion or a conversion may result in a frame shift generating an altered polypeptide; a substitution of at least one nucleotide may result in a premature stop codon, amino acid change or abnormal mRNA splicing; the deletion of several nucleotides, resulting in a deletion of one or more amino acids encoded by the nucleotides; the insertion of several nucleotides, such as by unequal recombination or gene conversion, resulting in an interruption of the coding sequence of a reading frame; duplication of all or a part of a sequence; transposition; or a rearrangement of a nucleotide sequence, as described in detail above. Such sequence changes alter the polypeptide encoded by an atherosclerosis susceptibility gene. For example, a nucleotide change resulting in a change in polypeptide sequence can alter the physiological properties of a polypeptide dramatically by resulting in altered activity, distribution and stability or otherwise affect on properties of a polypeptide. Alternatively, nucleotide sequence variants can result in changes affecting transcription of a gene or translation of its mRNA. A polymorphic site located in a regulatory region of a gene may result in altered transcription of a gene e.g. due to altered tissue specificity, altered transcription rate or altered response to transcription factors. A polymorphic site located in a region corresponding to the mRNA of a gene may result in altered translation of the mRNA e.g. by inducing stable secondary structures to the mRNA and affecting the stability of the mRNA. Such sequence changes may alter the expression of an atherosclerosis susceptibility gene.

The biomarkers for which we have disclosed novel associations with atherosclerosis, related clinical conditions, hypertension and obesity and type 2 diabetes have been known in prior art with their citations in human mitochondrial genome database MITOMAP (www.mitomap.org), which is a curated repository of data and a compendium of polymorphisms and mutations of the human mitochondrial DNA. Each biomarker has been linked to a specific map locus (Table 1) and to specific variable alleles present in a specific nucleotide position in the human mitochondrial genome, associated with mitochondrial DNA base substitution diseases (Table 2). Each biomarker has been also linked to specific variable alleles present in a specific nucleotide position in mitochondrial genome, and the nucleotide position has been specified with the nucleotide sequences flanking each SNP based on Revised Cambridge Reference Sequence (rCRS) of the Human Mitochondrial DNA (NCBI Reference Sequence NC_{—}012920.1 gi:251831106, GenBank) [26]. These biomarkers still have no official reference SNP (rs) ID identification tags assigned to each unique SNP by the National Center for Biotechnological Information (NCBI).

TABLE 1
Mitochondrial DNA function locations.

| Map Locus | Starting | Ending | Shorthand | Description |
|---|---|---|---|---|
| MT-RNR1 | 648 | 1601 | 12S | 12S ribosomal RNA |
| MT-TL1 | 3230 | 3304 | L(UUA/G) | tRNA leucine 1 |
| MT-ND1 | 3307 | 4262 | ND1 | NADH Dehydrogenase |
| | | | | subunit 1 |
| MT-TL2 | 12266 | 12336 | L(CUN) | tRNA leucine2 |
| MT-ND5 | 12337 | 14148 | ND5 | NADH dehydrogenase |
| | | | | subunit 5 |
| MT-ND6 | 14149 | 14673 | ND6 | NADH dehydrogenase |
| | | | | subunit 6 |
| MT-CYB | 14747 | 15887 | Cytb | Cytochrome b |

TABLE 2
Coding region sequence polymorphisms and reported
mitochondrial DNA base substitution diseases.

| Locus | Disease/State | Allele | Homoplasmy | Heteroplasmy |
|---|---|---|---|---|
| MT-RNR1 | DEAF | 1555 A→G | + | − |
| MT-TL2 | CPEO/KSS | 12315 G→A | − | + |
| MT-TL1 | MELAS | 3256 C→T | − | + |
| MT-ND5 | MELAS; Leigh | 13513 G→A | − | + |
| | Disease | | | |
| MT-CYB | Exercise | 14846 G→A | − | + |
| | Intolerance | | | |
| MT-CYB | MM | 15059 G→A | − | + |
| MT-ND1 | Neurogastro- | 3336 T→C | ? | ? |
| | intestinal | | | |
| | syndrome | | | |
| MT-ND6 | LDYT; Leigh | 14459 G→A | + | + |
| | Disease | | | |

Although the numerical chromosomal position of a heteroplasmy marker may still change upon annotating the current human genome build the heteroplasmy marker identification information such as variable alleles and flanking nucleotide sequences assigned to a heteroplasmy marker will remain the same. Those skilled in the art will readily recognize that the analysis of the nucleotides present in one or more heteroplasmy markers set forth in Table 3 of this invention in an individual's nucleic acid can be done by any method or technique capable of determining nucleotides present in a polymorphic site using the sequence information assigned in prior art to the rs IDs of the heteroplasmy markers listed in Table 3 of this invention. As it is obvious in the art the nucleotides present in polymorphisms can be determined from either nucleic acid strand or from both strands.

It is understood that the atherosclerosis, CHD, hypertension, obesity and type 2 diabetes associated heteroplasmy markers described in Table 3 of this invention may be associated with other polymorphisms and heteroplasmies in other loci. These other polymorphic sites associated with the heteroplasmy markers listed in Table 3 of this invention may be either equally useful as atherosclerosis biomarkers or even more useful as causative variations explaining the observed atherosclerosis association of the heteroplasmy markers of this invention.

The term “gene,” as used herein, refers to an entirety containing either an entire transcribed region and all regulatory regions of a gene or parts of these. The transcribed region of a gene including all exon and intron sequences of a gene including alternatively spliced exons and introns so the transcribed region of a gene contains in addition to polypeptide encoding region of a gene also regulatory and 5′ and 3′ untranslated regions present in transcribed RNA. Each gene has been assigned a specific and unique nucleotide sequence by the scientific community. By using the name of a gene those skilled in the art will readily find the nucleotide sequences of the corresponding gene and it's encoded mRNAs as well as amino acid sequences of it's encoded polypeptides although some genes may have been known with other name(s) in the art.

In certain methods described herein, an individual who has increased risk for developing atherosclerosis is an individual in whom one or more atherosclerosis associated polymorphisms selected from Table 3 of this invention are identified. In other embodiment also polymorphisms associated to one or more markers set forth in Table 3 may be used in risk assessment of atherosclerosis. The significance associated with an allele or a haplotype is measured by an odds ratio. In a further embodiment, the significance is measured by a percentage. In one embodiment, a significant risk is measured as odds ratio of 0.8 or less or at least about 1.2, including but not limited to: 0.1, 0.2, 0.3, 0.4, 0.5, 0.6, 0.7, 0.8, 1.2, 1.3, 1.4, 1.5, 1.6, 1.7, 1.8, 1.9, 2.0, 2.5, 3.0, 4.0, 5.0, 10.0, 15.0, 20.0, 25.0, 30.0 and 40.0. In a further embodiment, a significant increase or reduction in risk is at least about 20%, including but not limited to about 25%, 30%, 35%, 40%, 45%, 50%, 55%, 60%, 65%, 70%, 75%, 80%, 85%, 90%, 95% and 98%. In a further embodiment, a significant increase in risk is at least about 50%. It is understood however, that identifying whether a risk is medically significant may also depend on a variety of factors such as subject's family history of atherosclerosis, previously identified glucose intolerance, hypertriglyceridemia, hypercholesterolemia, elevated LDL cholesterol, low HDL cholesterol, elevated BP, hypertension, cigarette or other tobacco smoking, lack of physical activity, and inflammatory components as reflected by increased C-reactive protein levels or other inflammatory markers.

“Probes” or “primers” are oligonucleotides that hybridize in a base-specific manner to a complementary strand of nucleic acid molecules. By “base specific manner” is meant that the two sequences must have a degree of nucleotide complementarity sufficient for the primer or probe to hybridize to its specific target. Accordingly, the primer or probe sequence is not required to be perfectly complementary to the sequence of the template. Non-complementary bases or modified bases can be interspersed into the primer or probe, provided that base substitutions do not inhibit hybridization. The nucleic acid template may also include “non-specific priming sequences” or “nonspecific sequences” to which the primer or probe has varying degrees of complementarity. Probes and primers may include modified bases as in polypeptide nucleic acids (Nielsen P E et al, 1991). Probes or primers typically comprise about 15, to 30 consecutive nucleotides present e.g. in human genome and they may further comprise a detectable label, e.g., radioisotope, fluorescent compound, enzyme, or enzyme co-factor. Probes and primers to a heteroplasmy marker disclosed in Tables 1 and 2 are available in the art or can easily be designed using the flanking nucleotide sequences based on Revised Cambridge Reference Sequence (rCRS) of the Human Mitochondrial DNA and standard probe and primer design tools. Primers and probes for heteroplasmy markers disclosed in Table 3 can be used in risk assessment as well as molecular diagnostic methods and kits of this invention.

The invention comprises polyclonal and monoclonal antibodies that bind to a polypeptide related to one or more atherosclerosis associated heteroplasmy markers set forth in Table 3 of the invention. The term “antibody” as used herein refers to immunoglobulin molecules or their immunologically active portions that specifically bind to an epitope (antigen, antigenic determinant) present in a polypeptide or a fragment thereof, but does not substantially bind other molecules in a sample, e.g., a biological sample, which contains the polypeptide. Examples of immunologically active portions of immunoglobulin molecules include F(ab) and F(ab′) fragments which can be generated by treating the antibody with an enzyme such as pepsin. The term “monoclonal antibody” as used herein refers to a population of antibody molecules that are directed against a specific epitope and are produced either by a single clone of B cells or a single hybridoma cell line. Polyclonal and monoclonal antibodies can be prepared by various methods known in the art. Additionally, recombinant antibodies, such as chimeric and humanized monoclonal antibodies, comprising both human and non-human portions, can be produced by recombinant DNA techniques known in the art. Antibodies can be coupled to various enzymes, prosthetic groups, fluorescent materials, luminescent materials, bioluminescent materials, or radioactive materials to enhance detection.

An antibody specific for a polypeptide related to one or more atherosclerosis associated Heteroplasmy markers set forth in Table 3 of the invention can be used to detect the polypeptide in a biological sample in order to evaluate the abundance and pattern of expression of the polypeptide. Antibodies can be used diagnostically to monitor protein levels in tissue such as blood as part of a test predicting the susceptibility to atherosclerosis or as part of a clinical testing procedure, e.g., to, for example, determine the efficacy of a given treatment regimen.

“An atherosclerosis related condition” and “a CHD related condition” in the context of this invention comprise hypertension, obesity, dyslipidemias, the metabolic syndrome, insulin resistance, glucose intolerance, obesity and type 2 diabetes, clinical manifestations of CHD such as angina pectoris, myocardial infarction and sudden death, and complications of atherosclerosis or CHD such as retinopathy, nephropathy or neuropathy, coronary heart disease, cerebrovascular disease, congestive heart failure, intermittent claudication or another manifestation of arteriosclerosis. As Atherosclerosis is the most important risk factor and precursor of CHD, all examples and applications described in this invention concern, in addition to atherosclerosis, also CHD and CHD related conditions. For the sake of brevity, the word “atherosclerosis” is sometimes used to denote atherosclerosis related conditions.

### Diagnostic Methods and Test Kits

One major application of the current invention is diagnosing a susceptibility to atherosclerosis or related condition. The risk assessment methods and test kits of this invention can be applied to any healthy person as a screening or predisposition test, although the methods and test kits are preferably applied to high-risk individuals (subjects who have e.g. family history of atherosclerosis or related condition or elevated level of any atherosclerosis risk factor). Diagnostic tests that define genetic factors contributing to atherosclerosis might be used together with or independent of the known clinical risk factors to define an individual's risk relative to the general population. Better means for identifying those individuals susceptible for atherosclerosis should lead to better preventive and treatment regimens, including more aggressive management of the risk factors related to atherosclerosis and related diseases e.g. physicians may use the information on genetic risk factors to convince particular patients to adjust their life style e.g. to stop smoking, to reduce caloric intake, to make other changes in diet and to increase exercise.

In one embodiment of the invention, diagnosing a susceptibility to atherosclerosis in a subject, is made by detecting one or more heteroplasmy markers disclosed in Table 3 and FIGS. 1-16 of this invention in the subject's nucleic acid. An altered (high or low) level of heteroplasmy of atherosclerosis associated alleles of the assessed heteroplasmy markers (and haplotypes) in individual's genome indicates subject's increased risk for atherosclerosis and related conditions. The invention also pertains to methods of diagnosing a susceptibility to atherosclerosis in an individual comprising detection of a haplotype in an atherosclerosis risk gene that is more frequently present in an individual being atherosclerotic (affected), compared to the frequency of its presence in a healthy non-atherosclerotic individual (control), wherein the presence of the haplotype is indicative of a susceptibility to atherosclerosis. A haplotype may be associated with a reduced rather than increased risk of atherosclerosis, wherein the presence of the haplotype is indicative of a reduced risk of atherosclerosis. In other embodiment of the invention, diagnosis of susceptibility to atherosclerosis is done by detecting in the subject's nucleic acid one or more polymorphic sites being in linkage disequilibrium with one or more heteroplasmy markers and disclosed in Table 3 of this invention. Diagnostically the most useful polymorphic sites are those altering the biological activity of a polypeptide related to one or more atherosclerosis associated Heteroplasmy markers set forth in Table 3. Examples of such functional polymorphisms include, but are not limited to frame shifts, premature stop codons, amino acid changing polymorphisms and polymorphisms inducing abnormal mRNA splicing. Nucleotide changes resulting in a change in polypeptide sequence in many cases alter the physiological properties of a polypeptide by resulting in altered activity, distribution and stability or otherwise affect the properties of a polypeptide. Other diagnostically useful polymorphic sites are those affecting transcription of a gene or translation of it's mRNA due to altered tissue specificity, due to altered transcription rate, due to altered response to physiological status, due to altered translation efficiency of the mRNA and due to altered stability of the mRNA. Thus presence of nucleotide sequence variants altering the polypeptide structure and/or expression rate of a gene related to one or more atherosclerosis associated Heteroplasmy markers set forth in Table 3 of this invention in individual's nucleic acid is diagnostic for susceptibility to atherosclerosis.

In one embodiment of the invention, information of several heteroplasmies is combined to achieve improved prediction. For instance, ranked values (i.e. the numbers of fractiles, assigned according to interfractile borderlines) of percent of heteroplasmy for each mutation are summed up, keeping the sign (plus or minus) of beta coefficients obtained in multivariate statistical model (positive sign of coefficient value—addition, negative—subtraction). The resulting number may be called “mutational burden” as it combines the risk due to a number of heteroplasmies.

In diagnostic assays determination of the nucleotides present in one or more atherosclerosis associated heteroplasmy markers disclosed in this invention in an individual's nucleic acid can be done by any method or technique which can accurately determine nucleotides present in a polymorphic site. Numerous suitable methods have been described in the art (see e.g. references 27-32). These methods include, but are not limited to, hybridization assays, ASO-hybridization assays, restriction fragment length polymorphism assays, SSCP-analysis, ligation assays, primer extension assays, enzymatic cleavage assays, chemical cleavage assays and any combinations of these assays. The assays may or may not include PCR, real-time PCR, solid phase step, a microarray, modified oligonucleotides, labeled probes or labeled nucleotides, enzyme-linked immunosorbent assays, or sequencing and the assay may be multiplex or singleplex. As it is obvious in the art the nucleotides present in a polymorphic site can be determined from either nucleic acid strand or from both strands.

In another embodiment of the invention, a susceptibility to atherosclerosis is assessed from transcription products related to one or more atherosclerosis associated heteroplasmy markers set forth in Table 3 of this invention. Qualitative or quantitative alterations in transcription products can be assessed by a variety of methods described in the art, including e.g. hybridization methods, enzymatic cleavage assays, RT-PCR assays and microarrays. A test sample from an individual is collected and the said transcription products are assessed from RNA molecules present in the test sample and the result of the test sample is compared with results from atherosclerotic subjects (affected) and healthy non-atherosclerotic subjects (control) to determine individual's susceptibility to atherosclerosis.

The present invention particularly provides a method for risk assessment, diagnosis, subdiagnosis or prognosis of atherosclerosis, coronary heart disease (CHD) or an atherosclerosis or CHD related condition in a mammalian subject comprising:
a) providing a biological sample selected from the group consisting of a blood, saliva, urine, mucosal (such as a buccal swap), and hair shaft sample taken from the subject;
b) detecting one or more CHD and/or atherosclerosis or related phenotype associated biomarkers in said sample, wherein the biomarkers are related to one or more genes selected from the group consisting of MT-ND5, MT-RNR1, MT-TL1, MT-TL2, MT-ND1, MT-ND6 and MT-CYB genes, which encode subunit 5 of NADH dehydrogenase, 12S rRNA, tRNA-Leu 1, tRNA-Leu 2, subunits 1 and 6 of NADH dehydrogenase, and cytochrome B, respectively, or said biomarkers are related to one or more polypeptides encoded by said genes, and;
c) comparing the biomarker data from the subject to biomarker data from healthy and diseased people to make risk assessment, diagnosis, subdiagnosis or prognosis of atherosclerosis, CHD or a CHD related condition.

Preferably, the biomarkers in step b) of the above method are selected from the group consisting of 13513 G→A of the gene encoding subunit 5 of NADH dehydrogenase, 652 ins/del G and 1555 A→G of 12S rRNA gene, 3256 C→T of tRNA-Leu 1 gene, 3336 T→C of the gene encoding subunit 1 of NADH dehydrogenase, 12315 G→A of tRNA-Leu 2 gene, 14459 G→A of the gene encoding subunit 6 of NADH dehydrogenase, and 14846 G→A and 15059 G→A of cytochrome B gene. The most preferable marker is 13513 G→A of the gene encoding subunit 5 of NADH dehydrogenase.

In another embodiment of the invention, diagnosis of a susceptibility to atherosclerosis is made by examining expression, abundance, biological activities, structures and/or functions of polypeptides related to one or more atherosclerosis associated heteroplasmy markers disclosed in Table 3 of this invention. A test sample from an individual is assessed for the presence of alterations in the expression, biological activities, structures and/or functions of the polypeptides, or for the presence of a particular polypeptide variant (e.g., an isoform) related to one or more atherosclerosis associated heteroplasmy markers set forth in Table 3 of this invention. An alteration can be, for example, quantitative (an alteration in the quantity of the expressed polypeptide, i.e., the amount of polypeptide produced) or qualitative (an alteration in the structure and/or function of a polypeptide i.e. expression of a mutant polypeptide or of a different splicing variant or isoform). Alterations in expression, abundance, biological activity, structure and/or function of a atherosclerosis susceptibility polypeptide can be determined by various methods known in the art e.g. by assays based on chromatography, spectroscopy, colorimetry, electrophoresis, isoelectric focusing, specific cleavage, immunologic techniques and measurement of biological activity as well as combinations of different assays. An “alteration” in the polypeptide expression or composition, as used herein, refers to an alteration in expression or composition in a test sample, as compared with the expression or composition in a control sample and an alteration can be assessed either directly from the polypeptide itself or it's fragment or from substrates and reaction products of said polypeptide. A control sample is a sample that corresponds to the test sample (e.g., is from the same type of cells), and is from an individual who is not affected by atherosclerosis. An alteration in the expression, abundance, biological activity, function or composition of a polypeptide related to one or more atherosclerosis associated heteroplasmy markers set forth in Table 3 of this invention in the test sample, as compared with the control sample, is indicative of a susceptibility to atherosclerosis. In another embodiment, assessment of the splicing variant or isoform(s) of a polypeptide encoded by a polymorphic or mutant gene related to one or more atherosclerosis associated heteroplasmy markers set forth in Table 3 of this invention can be performed directly (e.g., by examining the polypeptide itself), or indirectly (e.g., by examining the mRNA encoding the polypeptide, such as through mRNA profiling).

Yet in another embodiment, a susceptibility to atherosclerosis can be diagnosed by assessing the status and/or function of biological networks and/or metabolic pathways related to one or more atherosclerosis associated heteroplasmy markers disclosed in Table 3. Status and/or function of a biological network and/or a metabolic pathway can be assessed e.g. by measuring amount or composition of one or several polypeptides or metabolites belonging to the biological network and/or to the metabolic pathway from a biological sample taken from a subject. Risk to develop atherosclerosis is evaluated by comparing observed status and/or function of biological networks and or metabolic pathways of a subject to the status and/or function of biological networks and or metabolic pathways of healthy and atherosclerotic subjects. Examples are the rRNA 12S, tRNA-Leu, cytochrome B system and NADH dehydrogenase and enzymes and other proteins interacting with these.

Another major application of the current invention is diagnosis of a molecular subtype of atherosclerosis in a subject. Molecular diagnosis methods and kits of this invention can be applied to a person being atherosclerotic. In one preferred embodiment, molecular subtype of atherosclerosis in an individual is determined to provide information of the molecular etiology of atherosclerosis. When the molecular etiology is known, better diagnosis and prognosis of atherosclerosis can be made and efficient and safe therapy for treating atherosclerosis in an individual can be selected on the basis of this subtype information. Physicians may use the information on genetic risk factors with or without known clinical risk factors to convince particular patients to adjust their life style and manage atherosclerosis risk factors and select intensified preventive and curative interventions for them. In other embodiment, biomarker information obtained from methods and kits for determining molecular subtype of atherosclerosis in an individual is for monitoring the effectiveness of their treatment. In one embodiment, methods and kits for determining molecular subtype of atherosclerosis are used to select human subjects for clinical trials testing atherosclerosis foods. The kits provided for diagnosing a molecular subtype of atherosclerosis in an individual comprise wholly or in part protocol and reagents for detecting one or more biomarkers and interpretation software for data analysis and atherosclerosis molecular subtype assessment.

The diagnostic assays and kits of the invention may further comprise a step of combining non-genetic information with the biomarker data to make risk assessment, diagnosis or prognosis of atherosclerosis. Useful non-genetic information comprises age, gender, smoking status, dietary information, physical activity, waist-to-hip circumference ratio (cm/cm), body mass index (kg/m²), the subject's family history of atherosclerosis and related conditions, previously identified or assessed glucose intolerance, diabetes, hypertriglyceridemia, high LDL cholesterol, low HDL cholesterol, elevated C-reactive protein (CRP), hypertension (HT) and elevated blood pressure (BP). The detection method of the invention may also further comprise a step determining blood, serum or plasma glucose, total cholesterol, HDL cholesterol, LDL cholesterol, triglyceride, apolipoprotein B and AI, fibrinogen, ferritin, transferrin receptor, C-reactive protein, insulin concentration or other CHD-risk associated biomarkers.

The score that predicts the probability of developing atherosclerosis may be calculated e.g. using a multivariate failure time model or a logistic regression equation. The results from the further steps of the method as described above render possible a step of calculating the probability of atherosclerosis using a logistic regression equation as follows. Probability of atherosclerosis=1/[1+e(−(−a+Σ(bi*Xi))], where e is the base for the natural logarithm, Xi are variables related to atherosclerosis, bi are coefficients of these variables in the logistic function, and a is the constant term in the logistic function, and wherein a and bi are preferably determined in the population in which the method is to be used, and Xi are preferably selected among the variables that have been measured in the population in which the method is to be used. Preferable values for bᵢ are between −20 and 20; and for i between 0 (none) and 100,000. A negative coefficient bᵢ implies that the marker is risk-reducing and a positive that the marker is risk-increasing. Xi are binary or quantitative variables that can have values or are coded as 0 (zero) or 1 (one) or any quantitative value such as heteroplasmy markers. The model may additionally include any interaction (product) or any polynomic terms of any variables Xi. An algorithm is developed for combining the information to yield a simple prediction of atherosclerosis as percentage of risk in one year, two years, five years, 10 years or 20 years. Alternative statistical models are failure-time models such as the Cox's proportional hazards' model, other iterative models and neural networking models.

Diagnostic test kits (e.g. reagent kits) of this invention comprise reagents, materials and protocols for assessing one or more biomarkers, and instructions and software for comparing the biomarker data from a subject to biomarker data from atherosclerotic and non-atherosclerotic people to make risk assessment, diagnosis or prognosis of atherosclerosis. Useful reagents and materials for kits comprise PCR primers, hybridization probes and primers as described herein (e.g., labeled probes or primers), allele-specific oligonucleotides, reagents for genotyping heteroplasmy markers, reagents for detection of labeled molecules, restriction enzymes (e.g., for RFLP analysis), DNA polymerases, RNA polymerases, DNA ligases, marker enzymes, antibodies which bind to polypeptides related to one or more atherosclerosis associated heteroplasmy markers disclosed in Table 3, means for amplification and/or nucleic acid sequence analysis of nucleic acid fragments containing one or more atherosclerosis associated heteroplasmy markers set forth in Table 3. In one embodiment, a kit for diagnosing susceptibility to atherosclerosis comprises primers and reagents for detecting the nucleotides present in one or more heteroplasmy markers selected from the Table 3 of this invention in individual's nucleic acid.

Yet another application of the current invention is related to methods and test kits for monitoring the effectiveness of a treatment for atherosclerosis. The disclosed methods and kits comprise taking a tissue sample (e.g. peripheral blood sample or adipose tissue biopsy) from a subject before starting a treatment, taking one or more comparable samples from the same tissue of the subject during the therapy, assessing expression (e.g., relative or absolute expression) of one or more genes related to one or more atherosclerosis associated heteroplasmy markers set forth in Table 3 of this invention in the collected samples of the subject and detecting differences in expression related to the treatment. Differences in expression can be assessed from mRNAs and/or polypeptides related to one or more atherosclerosis associated heteroplasmy markers set forth in Table 3 of this invention and an alteration in the expression towards the expression observed in the same tissue in healthy non-atherosclerotic individuals indicates the treatment is efficient. In a preferred embodiment the differences in expression related to a treatment are detected by assessing biological activities of one or more polypeptides related to one or more atherosclerosis associated heteroplasmy markers set forth in Table 3.

### EXPERIMENTAL SECTION

### Example 1

### Assessment of Atherosclerosis

Since the distribution of cIMT levels varies greatly between populations, and normal levels for particular population are usually unknown, they are best estimated separately for each population studied. For this purpose, we performed such study in Moscow, in which 885 apparently healthy persons (277 men and 608 women) free from manifested clinical atherosclerotic disease were involved.

To assess the atherosclerotic state of carotid arteries we used high-resolution B-mode ultrasonography with a linear vascular 7.5 MHz probe, SonoScape SSI-1000 scanner (China). The examination included the scanning of the left and right carotid arteries and the carotid sinus area, keeping a focus on the rear wall of the artery in the three fixed projections—anterolateral, lateral and posterolateral. The examination was carried out in a supine position after a 15-min rest. Measurements were made using M′Ath 3.1 software (IMT, France) at the site of the common carotid artery 10 mm long, opposite to the origin of the carotid sinus. The thickness of the intima-media layer of the posterior wall of the common carotid artery (cIMT) was defined as the distance from the leading edge of the first echogenic zone to the leading edge of the second echogenic zone. The mean of three measurements (in the anterolateral, lateral and posterolateral projections) was taken as an integral estimate of cIMT [24].

The reproducibility of IMT measurements was assessed according to the protocol of IMPROVE Study [24]. Briefly, ultrasound B-mode examination of carotids was performed independently by three operators in a random sample of 25 individuals at a day of admission to outpatient clinic and 10 days thereafter. Intima-media thickness measurement was performed by independent certified reader in blinded manner. Within-operator coefficients of variation (CV) was 2.6% for common carotid artery mean IMT, 3.3% for carotid bulb mean IMT, 4.8% for internal carotid artery mean IMT, and 4.2% for common carotid artery maximum IMT; reproducibility coefficients were 0.040, 0.062, 0.076, and 0.112, respectively. The between-operator coefficients of variation was 2.0% for common carotid artery mean IMT, 3.5% for carotid bulb mean IMT, 4.5% for internal carotid artery mean IMT, and 5.2% for common carotid artery maximum IMT and reproducibility coefficients 0.033, 0.089, 0.102, and 0.120, respectively.

The distributions of mean and maximum cIMT were defined in different age groups. Interquartile values were determined which allowed to distinguish persons predisposed or not predisposed to atherosclerosis. If a person belongs to the lowest quartile of age-adjusted cIMT distribution, he is regarded as non-predisposed to atherosclerosis; if he belongs to the highest quartile, then he has a predisposition to atherosclerosis. It is a way to select persons with extreme characteristics who are clearly different in the degree of susceptibility to atherosclerosis. The third group is also can be formed, which is a subgroup of predisposed persons but also with silent atherosclerotic plaques in the basin of carotid arteries. The following cut-off values for mean cIMT were defined in the Moscow population:

| | cIMT, μm | | | |
|---|---|---|---|---|
| | <50 | 51-60 | 61-70 | >70 |
| | years | years | years | years |
| Men, median | 750 | 810 | 900 | 930 |
| Predisposed to atherosclerosis | >800 | >910 | >995 | >1070 |
| Not predisposed to atherosclerosis | <660 | <740 | <830 | <850 |
| Women, median | 680 | 740 | 835 | 910 |
| Predisposed to atherosclerosis | >740 | >820 | >930 | >1015 |
| Not predisposed to atherosclerosis | <610 | <670 | <775 | <845 |

Totally, 156 participants free from manifested atherosclerotic disease were included in the study. Among them, 51 belonged to the lowest quartile of cIMT distribution, i.e. they had normal cIMT and were regarded as non-predisposed to atherosclerosis (NA group). Another 51 participants were in the highest quartile of cIMT distribution, i.e. they had abnormal cIMT and were regarded as predisposed to atherosclerosis (DIT group). The remaining 54 participants had atherosclerotic plaques along with abnormally elevated cIMT (AP group).

### Example 2

### DNA Extraction, Assessment of Mitochondrial Heteroplasmy and Statistical Analyses

Whole venous blood was taken from participants and used to isolate white blood cells, and the levels of heteroplasmy of different mitochondrial mutations were measured in DNA isolated from these cells.

Mitochondrial DNA was isolated with the Aquapure Genomic Tissue Kit by Bio-Rad according to the manufacturer's protocol. Fragments of mitochondrial DNA were obtained by polymerase chain reaction (PCR) followed by a pyrosequencing assay. The primers for the PCR are shown in Table 3 and the PCR conditions are shown in Table 4. Each 30 μl PCR reaction contained 0.4-0.6 μg mitochondrial DNA, 16.6 μM (NH₄)₂SO₄, 0.3 pM of each primer, 200 μM of each deoxyribonucleotriphosphate, 67 mM tris-HCl (pH 8.8), MgCl₂ (see Table 4), and 3 units of Taq-polymerase.

The quantitative proportion of mutant alleles was obtained by the pyrosequencing method [33-38], using the automated pyrosequencing device PSQ™ HS96MA. The pyrosequencing method is based on the measurement of the light intensity generated by the ATP-driven, luciferase-mediated conversion of luciferine to oxyluciferin. ATP is produced from pyrophosphate by ATP sulfurylase. The pyrophosphate is produced only when the added nucleotide complements the first unpaired base of the assayed biotinylated single strand DNA fragment. Therefore, the light intensity is proportional to the quantity of the complementary nucleotides incorporated into the DNA template. For example, if one cytosine (C) nucleotide were present in a certain position in the template, a peak corresponding to one portion of light would be seen on the pyrogram. If there were three C nucleotides, a triple peak would be observed. It should be noted that unlike other sequencing methods, pyrosequencing is designed for sequencing very small DNA fragments (5 to 20 nucleotides) containing the nucleotides of interest and some control neighboring nucleotides. The sequence analysis begins from the place of connection between the DNA sequencing primer and the PCR fragment.

The quantitative assay of mutant alleles was conducted by peak height analysis of the pyrogram in the studied domain of a single strand PCR fragment of the mitochondrial genome. It is important to note that a reverse primer should be used to analyze a DNA fragment that is complementary to the one assayed. Our aim was not to determine the homo- and heterozygosity for a mutation (which is typical for inherited mutations in the nuclear genome), but to estimate the heteroplasmy percentage of the studied mutation (which is more appropriate for the mitochondrial genome). This assay may be used in the clinical diagnostics for diseases associated with somatic mutations.

The heteroplasmy percentage in the DNA sample for each specific mutation was determined by analyzing the differences in peak sequence and size for homozygotes having 100% normal and 100% mutant alleles. The heteroplasmy percentage was calculated based on formula 1:

$$P = \frac{h - N}{M - N} \cdot 100 \% ,$$

where P is the heteroplasmy percentage;
h is the peak height for the studied nucleotide;
N is the peak height for the studied nucleotide corresponding to 100% of normal alleles in a sample;
M is the peak height for the studied nucleotide corresponding to 100% of mutant alleles in a sample.

We will consider the calculation of the heteroplasmy percentage for two types of mutations.

Type I

Point Replacement of One Nucleotide Pair by Another

Variant 1

The 3256C→T mutation will serve as an example. Since a reverse primer was used, we will analyze the guanine (G)→adenine (A) substitution (Table 3). When the mitochondrial genome contains 100% of a G nucleotide in position 3256, the size of peak A on the histogram will be 0, and the size of peak G will be 1. When there is 100% of an A nucleotide in position 3256, the size of peak A will be 1, and the size of peak G will be 0.

As an example, analyzing a DNA sample from a 29-year old man, we found that the size of peak A was 0.74, and the size of peak G was 2.12 on the practical pyrogram. The peak sum was 2.86, which corresponds to 1 unit on the theoretical pyrogram. Now we can calculate the heteroplasmy percentage for 3256C→T:

$$P = \frac{0.74 - 0}{2.86 - 0} \cdot 100 \% = 26 \%$$

Variant 2

The 13513G→A mutation will be considered (Table 3). With this single nucleotide substitution, the analyzed sequence consists not of two, but three or more nucleotide peaks. According to the theoretical pyrogram, the peak sizes at 100% of normal alleles with the G nucleotide in position 13513 (G/G) will be 1 A (peak 2), 1 G (peak 3), and 1 A (peak 4). At 100% mutant alleles with A in position 13513 (A/A), the peak sizes will be 3 A, 0 G, and 0 A. As seen from the pyrogram, the heteroplasmy percentage should be analyzed using peak 2, because the substitution occurs in that position. To calculate the heteroplasmy percentage, we sum up these three peaks and assume that they equal 3 units.

For example, analyzing a DNA sample from a 43-year old man, we found that the size of A (peak 2) was 12.03, that of G (peak 3) was 9.94, and that of A (peak 4) was 2.20. The peak sum was 24.17, which corresponds to 3 units on the theoretical pyrogram. It is necessary to first calculate the size of peak A that corresponds to 1 unit: 20.58/3=8.06. Subsequently the heteroplasmy percentage for 13513G→A can be calculated:

$$P = \frac{12.03 - 8.06}{24.17 - 8.06} \cdot 100 \% = 25 \%$$

Type II.

Deletion (Insertion) of Nucleotide Pairs

Variant 1

Deletion of One Nucleotide Pair

If one nucleotide were absent in the assayed single strand DNA fragment, the 100% normal DNA molecules will have an assayed nucleotide peak of n units, while the 100% mutant DNA molecules will have an assayed nucleotide peak of (n−1) units. A non-heteroplasmic peak from the same DNA fragment before or after the polymorphic fragment (designated as N in formula 1) is used as a control.

The heteroplasmy analysis for the 652delG mutation will be used as an example (Table 3). This mutation can be determined by the G nucleotide peak, which is the second peak in the analyzed sequence. According to the theoretical pyrogram, at 100% of normal genomes (G/G) the G peak is 2 units (FIG. 4a), and at 100% of genomes with the deletion (−/−) the G peak is 1 unit (FIG. 4b). The 5^{th} G peak with a height of 2 will be taken as a control peak.

For example, we will determine this mutation percentage in a DNA sample taken from a 50-year old man. The G peak height in the sample is 15.23. The control peak size is 18.56, which corresponds to 2 units on the theoretical pyrogram for the studied peak. Hence 1 unit of the studied G peak corresponds to a size of 9.28. The heteroplasmy percentage for 652delG is calculated as follows:

$$P = \frac{15.23 - 18.56}{9.28 - 18.56} \cdot 100 \% = 36 \%$$

Variant 2

Insertion of One Nucleotide Pair

The heteroplasmy percentage for this type of mutation is calculated in the same way as that of type I mutations, but at 100% alleles with an insertion, the size of the assayed peak is 1 unit more.

TABLE 3
Primers for PCR and pyrosequencing.

| Gene | Mutation | Direct primer for PCR | Reverse primer for PCR | Sequence primer |
|---|---|---|---|---|
| 12S rRNA | 652 | TAGACGGGCTCACATCAC | bio-GGGGTATCTAATCCCAGTTTGGGT | CCCATAAACAAATA |
| | ins/del G | (621-638) | (1087-1064) | (639-651) |
| | 1555 | TAGGTCAAGGTGTAGCCCATGAGGTGGCAA | bio-GTAAGGTGGAGTGGGTTTGGG | ACGCATTTATATAGAGGA |
| | A→G | (1326-1355) | (1704-1684) | (1537-1554) |
| tRNA-Leu | 3256 | bio-AGGACAAGAGAAATAAGGCC | ACGTTGGGGCCTTTGCGTAG | AAGAAGAGGAATTGA |
| (codon | C→T | (3129-3149) | (3422-3403) | (3300-3286) |
| recognizing | | | | |
| UUR) | | | | |
| subunit 1 | 3336 | bio-AGGACAAGAGAAATAAGGCC | ACGTTGGGGCCTTTGCGTAG | TGCGATTAGAATGGGTAC |
| of NADH | T→C | (3129-3149) | (3422-3403) | (3354-3337) |
| dehydrogenase | | | | |
| tRNA-Leu | 12315 | bio-CTCATGCCCCCATGTCTAA | TTACTTTTATTTGGAGTTGCAC | TTTGGAGTTGCAC |
| (codon | G→A | (12230-12249) | (12337-12317) | (1228-1216) |
| recognizing | | | | |
| CUN) | | | | |
| subunit 5 | 13513 | CCTCACAGGTTTCTACTCCAAA | bio-AAGTCCTAGGAAAGTGACAGCGAGG | AGGTTTCTACTCCAA |
| of NADH | G→A | (13491-13512) | (13825-13806) | (13497-13511) |
| dehydrogenase | | | | |
| subunit 6 | 14459 | CAGCTTCCTACACTATTAAAGT | bio-GTTTTTTTAATTTATTTAGGGGG | GATACTCCTCAATAGCCA |
| of NADH | G→A | (14303-14334) | (14511-14489) | (14439-14456) |
| dehydrogenase | | | | |
| cytochrome B | 14846 | CAGCTTCCTACACTATTAAAGT | bio-GTTTTTTTAATTTATTTAGGGGG | GCGCCAAGGAGTGA |
| | G→A | (14303-14334) | (14511-14489) | (14861-14848) |
| | 15059 | CAGCTTCCTACACTATTAAAGT | bio-GTTTTTTTAATTTATTTAGGGGG | TTTCTGAGTAGAGAAATGAT |
| | G→A | (14303-14334) | (14511-14489) | (15080-15061) |

TABLE 4
Conditions for the PCR of fragments of a mitochondrial genome.

| | Dimension of the | MgCl₂ concentration | Temperature | | |
|---|---|---|---|---|---|
| Mutation | PCR of fragment | in PCR buffer | Denaturation | Annealing | Extension |
| 652 ins/del G | 467 bp | 2.5 MM | 94° | 60° | 72° |
| 3256 C→T, 3336 T→C | 294 bp | | | 55° | |
| 13513 G→A | 335 bp | 1.5 MM | | | |
| 15059 G→A | 450 bp | | | | |
| 1555 A→G | 379 bp | 2.5 MM | | 50° | |
| 12315 G→A | 108 bp | | | | |
| 14459 G→A | 209 bp | 1.5 MM | | | |

### Example 3

### Associations of Leucocyte Mitochondrial Mutations with the Extent of Carotid Atherosclerosis

The level of heteroplasmy in human leukocytes was determined by pyrosequencing method adopted for conditions where both mutant and normal allele were present in the same specimen. The blood was taken from 156 persons in whom the extent of carotid atherosclerosis was determined by high-performance ultrasonography. This invention discloses the association of the selected mutations and genes in the mitochondrial genome with the extent of carotid atherosclerosis, CHD, hypertension and their complications in humans.

According to the ultrasonographic evaluation, 51 participants were non-atherosclerotic (NA), 51 had diffuse intima-media thickening (DIT), and the rest 54 had at least one atherosclerotic plaque in common carotid artery (AP). The level of heteroplasmy was significantly higher for C3256T, T3336C, G12315A and G15059A mutations in DIT and further in AP as compared to NA. On the opposite, the level of heteroplasmy declined from NA to AP for G13513A and Ins652G mutations. There was a strict linear-linear relationship between the extent of carotid atherosclerosis and quartiles of heteroplasmy for all above mutations (p<0.001 for C3256T, T3336C, G12315A, G15059A and G13513A, and p=0.002 for Ins652G). These results demonstrate that the mitochondrial genome is involved in the development of human atherosclerosis.

TABLE 5
The relationship between 10 leukocyte mtDNA heteroplasmies (in quartiles)
and the extent of carotid atherosclerosis (NA, DIT and PA).

| | Mean percent of heteroplasmy¹ | | | | | |
|---|---|---|---|---|---|---|
| | Evidently normal | Abnormal diffuse | Abnormal diffuse intima- | Statistics² | | |
| | thickness of intima- | intima-media | media thickening + | Linear-by- | | |
| | media complex (NA), | thickening | atherosclerotic plaque | linear | | Spearman's |
| Mutation | n = 51 | (DIT), n = 51 | (AP), n = 54 | relationship | Gamma | Rho |
| 652 del G | 3.1 ± 1.4 (9.7) | 1.3 ± 0.6 (4.1) | 4.8 ± 1.3 (9.7) | 2.2 | 0.24 ± 0.16 | 0.12 ± 0.08 |
| | | p = 0.40 vs N | p = 0.13 vs N | p = 0.14 | p = 0.15 | p = 0.13 |
| | | | p = 0.019 vs DIT | | | |
| 652 ins G | 18.8 ± 2.3 (16.8) | 20.1 ± 2.0 (14.6) | 9.3 ± 1.4 (10.5) | 9.4 | 0.29 ± 0.09 | 0.25 ± 0.08 |
| | | p = 0.43 vs N | p = 0.002 vs N | p = 0.002 | p = 0.001 | p = 0.002 |
| | | | p < 0.001 vs DIT | | | |
| 1555 A→G | 21.8 ± 1.6 (11.3) | 10.6 ± 0.3 (1.9) | 15.4 ± 0.5 (4.0) | 0.3 | 0.03 ± 0.10 | 0.04 ± 0.09 |
| | | p < 0.001 vs N | P = 0.056 vs N | p = 0.57 | p = 0.80 | p = 0.60 |
| | | | p < 0.001 vs DIT | | | |
| 3256 C→T | 15.8 ± 0.4 (2.6) | 17.7 ± 0.6 (4.4) | 44.6 ± 1.1 (8.4) | 94.4 | 0.82 ± 0.05 | 0.77 ± 0.04 |
| | | p = 0.025 vs N | p < 0.001 vs N | p < 0.001 | p < 0.001 | p < 0.001 |
| | | | p < 0.001 vs DIT | | | |
| 3336 T→C | 5.0 ± 0.3 (2.2) | 7.8 ± 0.3 (2.5) | 12.0 ± 0.8 (5.5) | 67.3 | 0.77 ± 0.05 | 0.66 ± 0.05 |
| | | p < 0.001 vs N | p < 0.001 vs N | p < 0.001 | p < 0.001 | p < 0.001 |
| | | | p < 0.001 vs DIT | | | |
| 12315 G→A | 24.7 ± 1.3 (9.4) | 27.8 ± 1.0 (7.0) | 57.4 ± 1.2 (9.1) | 79.8 | 0.79 ± 0.05 | 0.72 ± 0.05 |
| | | p = 0.016 vs N | p < 0.001 vs N | p < 0.001 | p < 0.001 | p < 0.001 |
| | | | p < 0.001 vs DIT | | | |
| 13513 G→A | 33.1 ± 1.4 (9.7) | 20.1 ± 1.2 (8.3) | 5.7 ± 0.7 (5.2) | 106.3 | 0.93 ± 0.02 | 0.83 ± 0.03 |
| | | p < 0.001 vs N | p < 0.001 vs N | p < 0.001 | p < 0.001 | p < 0.001 |
| | | | p < 0.001 vs DIT | | | |
| 14459 G→A | 15.8 ± 0.4 (2.6) | 34.1 ± 1.9 (13.7) | 13.4 ± 0.8 (6.0) | 1.4 | 0.10 ± 0.08 | 0.10 ± 0.09 |
| | | p < 0.001 vs N | p = 0.37 vs N | p = 0.24 | p = 0.24 | p = 0.20 |
| | | | p < 0.001 vs DIT | | | |
| 14846 G→A | 8.9 ± 0.4 (2.5) | 29.5 ± 3.5 (25.3) | 10.7 ± 1.2 (8.6) | 0.8 | 0.08 ± 0.09 | 0.06 ± 0.09 |
| | | p < 0.001 vs N | P = 0.87 vs N | p = 0.36 | p = 0.38 | p = 0.48 |
| | | | p < 0.001 vs DIT | | | |
| 15059 G→A | 26.0 ± 1.0 (7.1) | 48.3 ± 1.1 (7.8) | 43.5 ± 1.1 (8.4) | 52.3 | 0.60 ± 0.07 | 0.59 ± 0.06 |
| | | p < 0.001 vs N | p < 0.001 vs N | p < 0.001 | p < 0.001 | p < 0.001 |
| | | | p = 0.006 vs DIT | | | |

¹The significance of differences is estimated by Mann-Whitney U-test for independent samples, mean values and SEM are indicated (SD in parentheses)
²The relationship between the extent of carotid atherosclerosis (NA, DIT and PA) and quartiles of heteroplasmy (1^{st}, 2^{nd}, 3^{rd}, and 4^{th} - see Table 4 for distribution statistics and interquartile borderlines) is estimated by contingency table with linear-linear relationship coefficient, gamma coefficient and Pearson's correlation estimates.

TABLE 6
Descriptive statistics and interquartile limits (cut-offs) for the
distribution of the percent of heteroplasmy in 156 study subjects.

| | | 13513 | 14459 | 3256 | 3336 | 652 | 652 | 15059 | 12315 | 1555 | 14846 |
|---|---|---|---|---|---|---|---|---|---|---|---|
| | | G→A | G→A | C→T | T→C | del G | ins G | G→A | G→A | A→G | G→A |
| N | Valid | 156 | 156 | 156 | 156 | 156 | 156 | 156 | 156 | 156 | 156 |
| | Missing | 0 | 0 | 0 | 0 | 0 | 0 | 0 | 0 | 0 | 0 |
| Mean | | 19.4 | 20.4 | 26.4 | 8.3 | 3.1 | 16.0 | 39.3 | 37.0 | 15.9 | 16.2 |
| Std. Error of Mean | | 1.1 | 1.1 | 1.2 | 0.4 | 0.7 | 1.2 | 1.0 | 1.4 | 0.7 | 1.4 |
| Median | | 17.0 | 16.5 | 20.0 | 8.0 | 0.0 | 14.0 | 41.0 | 30.0 | 13.0 | 11.0 |
| Std. Deviation | | 13.8 | 13.2 | 14.5 | 4.8 | 8.4 | 14.9 | 12.3 | 17.2 | 8.3 | 17.9 |
| Minimum | | 0 | 6 | 10 | 0 | 0 | 0 | 4 | 11 | 0 | 4 |
| Maximum | | 55 | 82 | 74 | 44 | 54 | 81 | 65 | 80 | 43 | 96 |
| Percentiles | 25 | 6.3 | 11.0 | 15.0 | 5.0 | 0.0 | 0.0 | 30.0 | 25.0 | 11.0 | 8.0 |
| | 50 | 17.0 | 16.5 | 20.0 | 8.0 | 0.0 | 14.0 | 41.0 | 30.0 | 13.0 | 11.0 |
| | 75 | 33.8 | 28.0 | 39.0 | 10.8 | 0.0 | 26.0 | 47.0 | 53.8 | 18.0 | 16.0 |

Regression analysis was performed, which has demonstrated that the extent of atherosclerosis (ranked values NA=1, DIT=2, PA=3) was dependent of the ranked values of heteroplasmy of the above mitochondrial mutations. The regression model, which included all markers, had adjusted R²=0.886, p<0.001 by ANOVA, however beta coefficients for several mutations did not reach statistical significance. After excluding the less significant mutations in a step-by-step procedure, a minimal regression model was constructed, in which the extent of atherosclerosis was highly dependent of heteroplasmy of four markers (13513 G→A, 3256 C→T, 15059 G→A, and 12315 G→A). Standardized beta coefficients were −0.408, 0.273, 0.290 and 0.235, respectively; p<0.001 for all. The regression constant was 1.340±0.146, p<0.001. For this model, R=0.942, R²=0.888, adjusted R²=0.885, p<0.001 by ANOVA.

Thus, seven genetic markers were linearly related to the severity of carotid atherosclerosis, and four of them remained significant in the linear step-up regression model with p<0.001 for beta-coefficients. Moreover, they were also significantly correlated at p<0.001 between each other, suggesting the presence of linkage disequilibrium.

Ranked values (i.e. the numbers of quartiles, assigned according to interquartile cut-offs as defined in Table 6) of percent of heteroplasmy for each mutation were summed up keeping the sign (plus or minus) of beta coefficients obtained in linear regression model (positive sign of coefficient value—addition, negative—subtraction). The resulting number was called “mutational burden” (taken out of all 11 markers investigated). The association of mutational burden with the extent of carotid atherosclerosis is shown in the FIG. 1.

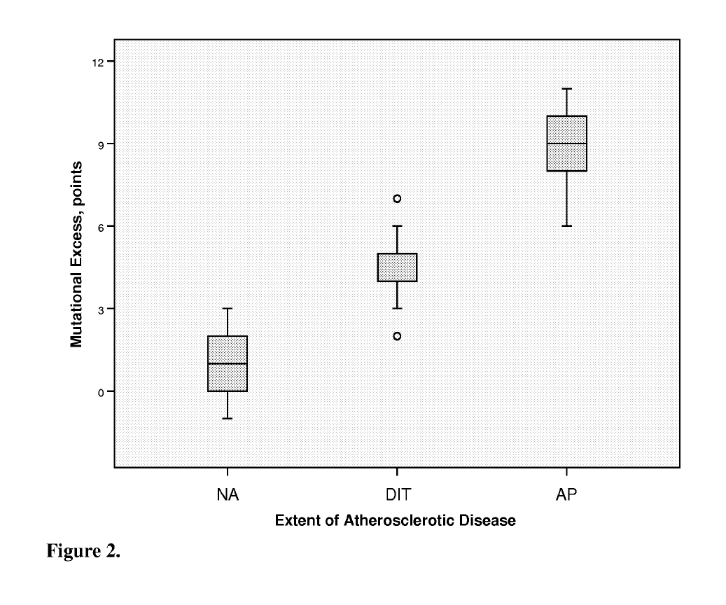

The FIG. 2 shows an association of the extent of carotid atherosclerosis with only those 4 markers, which were associated with the degree of atherosclerosis in the linear step-up regression model with p<0.001. In this case, the sum of ranked values was called “mutational excess”. In both graphs the circles define outliners.

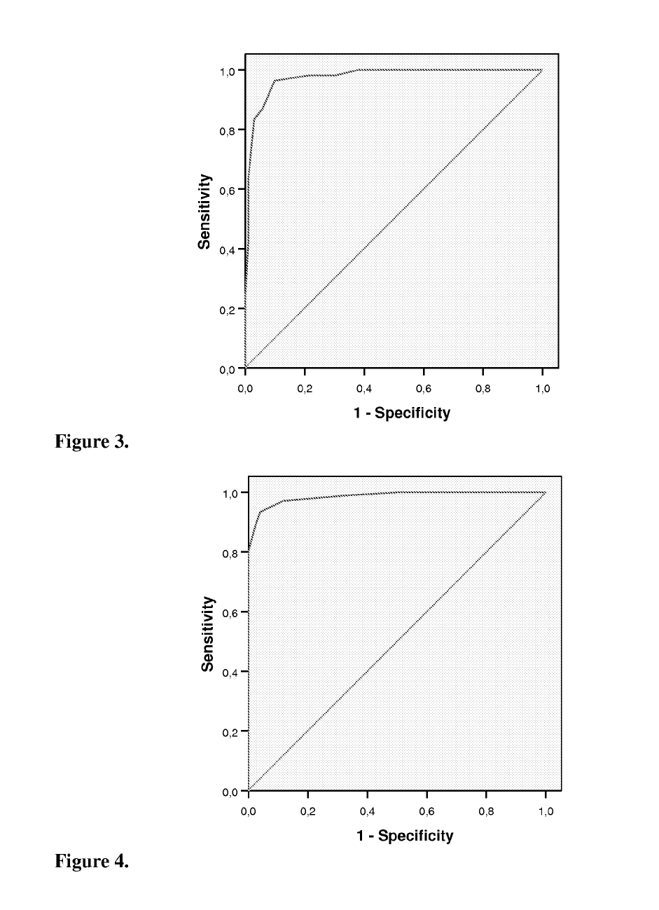

FIGS. 3-6 present receive-operator curves (ROC) for mutational burden or mutational excess, which were calculated as explained above.

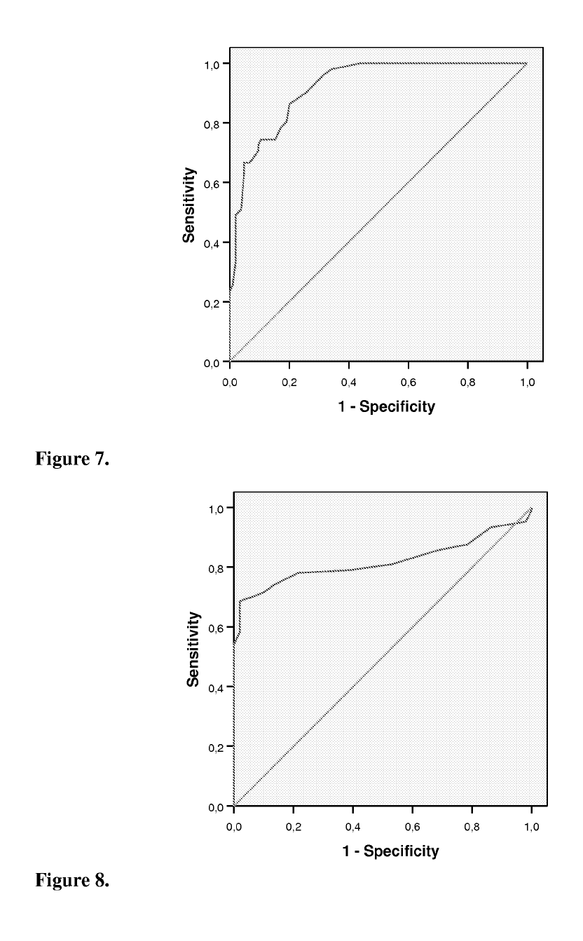

The last series of graphs (FIGS. 7-17) show receive-operator curves for absolute values of heteroplasmy for separate mutations, which were found to be associated with the extent of carotid atherosclerosis.

### Example 4

### Associations of mtDNA Heteroplasmies with Clinical Outcomes

In 192 participants of the study in Moscow, the mutational burden (i.e. the sum of quartile numbers of the 10 markers of Table 3) was significantly associated with any coronary heart disease (CHD), as compared with subjects with no CHD. The area under ROC was 0.67 (95% CI 0.57-0.77), p=0.001, taking into account the plus-minus signs. The heteroplasmies 3256C→T, 3336T→C, 12315G→A, 13513G→A and 14459G→A were significantly associated with prevalent CHD in 192 subjects from Moscow (Table 7).

TABLE 7
The association between 10 leukocyte mtDNA
heteroplasmies (%) and the presence of CHD.

| | Heteroplasmy, % | | | | | |
|---|---|---|---|---|---|---|
| mtDNA | CHD absent | | CHD present | | P | P Mann- |
| mutation | n = 147 | | n = 45 | | student | Whitney |
| 652delG | 3.8 | (13.6) | 1.7 | (4.9) | 0.11 | 0.16 |
| 652insG | 20.4 | (18.4) | 20.6 | (18.6) | 0.93 | 0.94 |
| 1555A→G | 16.8 | (11.3) | 15.5 | (9.5) | 0.45 | 0.87 |
| 3256C→T | 21.6 | (13.4) | 28.8 | (17.3) | 0.004 | 0.031 |
| 3336T→C | 7.9 | (8.7) | 9.6 | (6.7) | 0.17 | 0.019 |
| 12315G→A | 30.9 | (18.9) | 38.5 | (20.2) | 0.028 | 0.030 |
| 13513G→A | 25.3 | (18.4) | 18.8 | (19.2) | 0.048 | 0.006 |
| 14459G→A | 29.0 | (21.9) | 21.4 | (17.3) | 0.034 | 0.018 |
| 14846G→A | 16.1 | (17.5) | 14.7 | (17.2) | 0.63 | 0.31 |
| 15059G→A | 37.6 | (16.8) | 37.8 | (16.8) | 0.94 | 0.96 |

Of the tested 10 leukocyte mtDNA heteroplasmies, 1555A→G, 12315G→A, 13513G→A, and 14846G→A were significantly associated with prevalent MI (Table 8).

TABLE 8
The association between 10 leukocyte mtDNA heteroplasmies
(%) and the History of myocardial infarction (MI).

| | Heteroplasmy, % | | | | | |
|---|---|---|---|---|---|---|
| mtDNA | AMI absent | | AMI present | | P | P Mann- |
| mutation | n = 185 | | n = 7 | | student | Whitney |
| 652delG | 3.28 | (15.3) | 4.0 | (6.9) | 0.80 | 0.45 |
| 652insG | 20.7 | (18.4) | 13.4 | (19.0) | 0.36 | 0.24 |
| 1555A→G | 16.7 | (11.1) | 11.7 | (3.5) | 0.009 | 0.29 |
| 3256C→T | 22.7 | (14.2) | 38.4 | (20.7) | 0.005 | 0.041 |
| 3336T→C | 8.3 | (8.4) | 9.6 | (3.3) | 0.36 | 0.13 |
| 12315G→A | 32.1 | (19.3) | 48.0 | (18.5) | 0.033 | 0.043 |
| 13513G→A | 24.2 | (18.8) | 12.9 | (12.3) | 0.052 | 0.07 |
| 14459G→A | 27.4 | (21.2) | 22.9 | (17.9) | 0.54 | 0.70 |
| 14846G→A | 16.1 | (17.6) | 8.7 | (4.1) | 0.002 | 0.07 |
| 15059G→A | 37.6 | (16.9) | 37.6 | (12.9) | 0.99 | 0.88 |

Of the tested 10 leukocyte mtDNA heteroplasmies, 3256C→T, 14459G→A and 15059G→A were significantly associated with prevalent hypertension (Table 9).

TABLE 9
The association between 10 leukocyte mtDNA heteroplasmies
(%) and the prevalence of hypertension (HT).

| | Heteroplasmy, % | | | | | |
|---|---|---|---|---|---|---|
| mtDNA | HT absent | | HT present | | P | P Mann- |
| mutation | n = 67 | | n = 125 | | student | Whitney |
| 652delG | 3.0 | (12.8) | 3.5 | (11.8) | 0.80 | 0.88 |
| 652insG | 22.8 | (20.2) | 19.2 | (17.4) | 0.22 | 0.25 |
| 1555A→G | 17.8 | (13.3) | 15.8 | (9.4) | 0.22 | 0.70 |
| 3256C→T | 20.2 | (13.0) | 24.9 | (15.3) | 0.035 | 0.07 |
| 3336T→C | 7.0 | (4.2) | 9.0 | (9.8) | 0.054 | 0.08 |
| 12315G→A | 29.6 | (18.0) | 34.3 | (20.0) | 0.11 | 0.19 |
| 13513G→A | 25.1 | (17.4) | 23.0 | (19.4) | 0.46 | 0.17 |
| 14459G→A | 30.8 | (23.1) | 25.4 | (19.8) | 0.09 | 0.15 |
| 14846G→A | 15.9 | (18.6) | 15.8 | (16.8) | 0.98 | 0.62 |
| 15059G→A | 34.6 | (16.6) | 39.2 | (16.7) | 0.07 | 0.14 |

Of the tested 10 leukocyte mtDNA heteroplasmies, 3336T→C and 14846G→A were significantly associated with prevalent type 2 diabetes (Table 10).

TABLE 10
The association between 10 leukocyte mtDNA heteroplasmies
(%) and the prevalence of type 2 diabetes (DM).

| | Heteroplasmy, % | | | | | |
|---|---|---|---|---|---|---|
| mtDNA | DM absent | | DM present | | P | P Mann- |
| mutation | n = 168 | | n = 24 | | student | Whitney |
| 652delG | 2.9 | (10.4) | 6.3 | (20.7) | 0.20 | 0.73 |
| 652insG | 20.7 | (18.6) | 18.4 | (17.6) | 0.55 | 0.58 |
| 1555A→G | 16.9 | (11.3) | 14.1 | (6.8) | 0.10 | 0.34 |
| 3256C→T | 22.7 | (14.4) | 27.0 | (16.4) | 0.23 | 0.34 |
| 3336T→C | 7.7 | (5.0) | 12.3 | (19.2) | 0.012 | 0.18 |
| 12315G→A | 32.1 | (19.3) | 36.9 | (20.2) | 0.28 | 0.32 |
| 13513G→A | 24.1 | (18.2) | 21.3 | (22.7) | 0.57 | 0.15 |
| 14459G→A | 26.5 | (20.5) | 32.6 | (24.7) | 0.25 | 0.32 |
| 14846G→A | 15.2 | (17.0) | 20.0 | (19.5) | 0.27 | 0.050 |
| 15059G→A | 37.2 | (16.7) | 40.2 | (17.5) | 0.44 | 0.50 |

Of the tested 10 leukocyte mtDNA heteroplasmies, 14459G→A was significantly associated with obesity as defined BMI over 30 kg/m² (Table 11).

TABLE 11
The association between 10 leukocyte mtDNA heteroplasmies
(%) and the prevalence of obesity.

| | Heteroplasmy, % | | | | | |
|---|---|---|---|---|---|---|
| mtDNA | OB present | | OB absent | | P | P Mann- |
| mutation | n = 37 | | n = 155 | | student | Whitney |
| 652delG | 4.2 | (10.7) | 3.1 | (12.5) | 0.57 | 0.58 |
| 652insG | 18.6 | (15.6) | 20.9 | (19.0) | 0.44 | 0.41 |
| 1555A→G | 16.3 | (9.0) | 16.6 | (11.3) | 0.90 | 0.88 |
| 3256C→T | 25.8 | (15.1) | 22.7 | (14.6) | 0.26 | 0.16 |
| 3336T→C | 7.4 | (4.0) | 8.5 | (9.0) | 0.26 | 0.72 |
| 5178C→A | 15.7 | (6.1) | 15.6 | (11.5) | 0.94 | 0.30 |
| 12315G→A | 34.9 | (18.1) | 32.1 | (19.7) | 0.41 | 0.34 |
| 13513G→A | 20.1 | (13.7) | 24.6 | (19.7) | 0.11 | 0.42 |
| 14459G→A | 21.0 | (17.2) | 28.7 | (21.7) | 0.044 | 0.013 |
| 14846G→A | 16.1 | (19.2) | 15.7 | (17.0) | 0.92 | 0.98 |
| 15059G→A | 38.4 | (12.6) | 37.4 | (17.6) | 0.76 | 0.77 |

There were also significant associations with angina pectoris (area under ROC 0.65, 95% CI 0.55-0.75, p=0.002), myocardial infarction (MI) (area under ROC 0.80, 95% CI 0.72-0.89, p=0.012). There were also suggestive associations with cerebrovascular stroke (area under ROC 0.64), hypertension (area under ROC 0.56) and obesity and type 2 diabetes (area under ROC 0.62, p=0.075).

### Example 5

### Known Associations of the mtDNA Markers with Clinical Conditions

Several of the 10 markers of Table 3 are known in the art to have a role in a number of clinical conditions. A short summary of these is presented below.

652 Ins/DelG induces the damage of coding region of MT-RNR1 gene encoding 12S RNA, and is associated with mitochondrial myopathy.

1555 A→induces the damage of coding region of MT-RNR1 gene encoding 12S RNA, and is associated with deafness, increased sensitivity to aminoglycosides.

3256 C→T induces the damage of coding region of MT-TL1 gene encoding tRNA-leucine (UUR recognizing codone), affects the transport of leucine, and is associated with neurodegenerative diseases, encephalopathy, lactoacidosis, myopathy, cardiomyopathy, strokes of right parietooccipitalis regions, and oxidative defects of muscular metabolism. 3336 T→C induces the damage of coding region of MT-ND1 gene encoding subunit 1 of NADH dehydrogenase, thus affecting the catalysis of NADH oxidation and CoQ (ubiquinone) reduction, and is associated with obesity and type 2 diabetes mellitus.

12315 G→A induces the damage of coding region of MT-TL2 gene encoding tRNA-leucine (CUN recognizing codone), affects the transport of leucine, and is associated with progressive ophtalmoplegy, blepharoptosis, neurosensoric deafness, pigmental retinopathy, and weakness of extremities.

13513 G→A induces the damage of coding region of MT-ND5 gene encoding subunit 5 of NADH dehydrogenase, thus affecting the catalysis of NADH oxidation and CoQ (ubiquinone) reduction, and is associated with hereditary encephalomyopathy, cardiomyopathy and WPW syndrome.

14459 G→A induces the damage of coding region of MT-ND6 gene encoding subunit 6 of NADH dehydrogenase, thus affecting the catalysis of NADH oxidation and CoQ (ubiquinone) reduction, results in alanine to valine substitution in conserved region of ND6 protein at position 72, and is associated with hereditary ocular neuropathy, atrophy of visual nerve, Leber's hereditary visual neuropathy, dysfunction of basal ganglia, musculospastic syndrome and encephalopathy.

14846 G→A induces the damage of coding region of MT-CYB gene encoding cytochrome B, results in glycine to serine substitution in position 34, thus affecting intermediate transfer of electrons in mitochondrial respiratory chains, reducing enzymatic function of cytochrome B, and associated with mitochondrial myopathies.

15059 G→A induces the damage of coding region of MT-CYB gene encoding cytochrome B, results in glycine to stop codone substitution at position 190, thus stopping translation and leading to the loss of 244 amino acids at C-terminal of protein, reducing enzymatic function of cytochrome B, and associated with mitochondrial myopathies.

### REFERENCES

1. Andreu A L, Hanna M G, Reichmann H et al. Exercise intolerance due to mutations in the cytochrome b gene of mitochondrial DNA. N Engl J. Med. 1999:341; 1037-44.
2. Chol M, Lebon S, Bénit P et al. The mitochondrial DNA G13513A MELAS mutation in the NADH dehydrogenase 5 gene is a frequent cause of Leigh-like syndrome with isolated complex I deficiency. J Med. Genet. 1003; 40:188-91.
3. Han C B, Ma J M, Xin Y et al. Mutations of mitochondrial 12S rRNA in gastric carcinoma and their significance. World J Gastroenterol. 2005; 11:31-5.
4. Tanimoto H, Nishio H, Matsuo M, Nibu K. A novel mitochondrial mutation, 1556C-->T, in a Japanese patient with streptomycin-induced tinnitus. Acta Otolaryngol. 2004; 124:258-61.
5. Yamagata K, Muro K, Usui J et al. Mitochondrial DNA mutations in focal segmental glomerulosclerosis lesions. J Am Soc Nephrol. 2002; 13:1816-23.
6. Matsunaga H, Tanaka Y, Tanaka M, et al. Antiatherogenic mitochondrial genotype in patients with obesity and type 2 diabetes. Diabetes Care. 2001; 24:500-3.
7. Wong L J, Chen T J, Tan D J. Detection of Mitochondrial DNA Mutations by Temporal Temperature Gradient Gel Electrophoresis. Electrophoresis. 2004; 25:2602-10.
8. Fu K, Hartlen R, Johns T et al. A novel heteroplasmic tRNAleu(CUN) mtDNA point mutation in a sporadic patient with mitochondrial encephalomyopathy segregates rapidly in skeletal muscle and suggests an approach to therapy. Hum Mol Genet. 1996; 5:1835-40.
9. Solano A, Roig M, Vives-Bauza C et al. Bilateral striatal necrosis associated with a novel mutation in the mitochondrial ND6 gene. Ann Neurol. 2003; 54:527-30.
10. Horváth R, Scharfe C, Hoeltzenbein M, et al. Childhood onset mitochondrial myopathy and lactic acidosis caused by a stop mutation in the mitochondrial cytochrome C oxidase III gene. J Med Genet. 2002; 39:812-6.
11. Kim D-S, Jung D-S, Park K-H et al. Histochemical and molecular genetic study of MELAS and MERF in korean patients. J Korean Med Scien. 2002; 17:103-12.
12. Varlamov D A, Kudin A P, Vielhaber S et al. Metabolic consequences of a novel missense mutation of the mtDNA CO I gene. Hum Mol Genet. 2002; 11:1797-805.
13. Gropman A, Chen T J, Perng C L et al. Variable clinical manifestation of homoplasmic G14459A mitochondrial DNA mutation. Am J Med Genet A. 2004; 124:377-82.
14. Man P Y, Griffiths P G, Brown D T et al. The epidemiology of Leber hereditary optic neuropathy in the North East of England. Am J Hum Genet. 2003; 72:333-9.
15. Nishigaki Y, Marti R, Copeland W C, Hirano M. Site-specific somatic mitochondrial DNA point mutations in patients with thymidine phosphorylase deficiency. J Clin Invest. 2003; 111:1913-21.
16. Baracca A, Barogi S, Carelli V, Lenaz G, Solaini G. Catalytic activities of mitochondrial ATP synthase in patients with mitochondrial DNA T8993G mutation in the ATPase 6 gene encoding subunit a. J Biol Chem. 2000; 275:4177-82.
17. Jeppesen T D, Schwartz M, Hansen K et al. Late onset of stroke-like episode associated with a 3256C-->T point mutation of mitochondrial DNA. J Neurol Sci. 2003; 214:17-20.
18. Wallace D C. Mitochondrial DNA sequence variation in human evolution and disease. Proc Natl Acad Sci USA. 1994; 91:8739-46.
19. Sazonova M, Budnikov E, Khasanova Z, Sobenin I, Postnov A, Orehov A. Studies of the human aortic intima by a direct quantitative assay of mutant alleles in the mitochondrial genome. Atherosclerosis 2009; 204:184-90.
20. Takagi K, Yamada Y, Gong J-S, Sone T, Yokota M, Tanaka M. Association of a 5178C>A (Leu237Met) polymorphism in the mitochondrial DNA with a low prevalence of myocardial infarction in Japanese individuals. Atherosclerosis 2004; 175:281-6.
21. Stein J H, Korcarz C E, Hurst R T et al. (2008) Use of carotid ultrasound to identify subclinical vascular disease and evaluate cardiovascular disease risk: a consensus statement from the American Society of Echocardiography Carotid Intima-Media Thickness Task Force. Endorsed by the Society for Vascular Medicine. J Am Soc Echocardiogr. 21:93-111.
22. Salonen J T, Salonen R. Ultrasound B-mode imaging in observational studies of atherosclerotic progression. Circulation 1993; 87 (suppl. II):55-65.
23. Amato M., Montrosi P., Ravani A. et al. Carotid intima-media thickness by B-mode ultrasound as surrogate of coronary atherosclerosis: correlation with quantitative coronary angiography and coronary intravascular ultrasound findings. Eur Heart J. 2007:28; 2094-101.
24. Baldassarre D, Nyyssönen K, Rauramaa R. et al. Cross-sectional analysis of baseline data to identify the major determinants of carotid intima-media thickness in a European population: the IMPROVE study. Eur Heart J. 2010 31:614-22.
25. Salonen J T, Ylä-Herttuala S, Yamamoto R, Butler S, Korpela H, Salonen R, Nyyssönen K, Palinski W, Witztum J L. Autoantibody against oxidised LDL and progression of carotid atherosclerosis. Lancet 1992; 339:883-7.
26. Andrews R M, Kubacka I, Chinnery P F, Lightowlers R N, Turnbull D M, Howell N. Reanalysis and revision of the Cambridge reference sequence for human mitochondrial DNA. Nat Genet. 1999; 23:147
27. Yoo J H, Suh B, Park T S, Shin M G, Choi Y D, Lee C H, Choi J R. Analysis of fluorescence in situ hybridization, mtDNA quantification, and mtDNA sequence for the detection of early bladder cancer. Cancer Genet Cytogenet. 2010; 198:107-17
28. Wong L J, Cobb B R, Chen T J. Molecular analysis of mitochondrial DNA point mutations by polymerase chain reaction. Methods Mol Biol. 2006; 336:135-43.
29. Hiratsuka M, Ebisawa A, Sakuyama K, Matsubara Y, Kure S, Soya Y, Konno Y, Sasaki T, Kishiba A, Mizugaki M. Competitive allele-specific short oligonucleotide hybridization (CASSOH) with enzyme-linked immunosorbent assay (ELISA) for the detection of pharmacogenetic single nucleotide polymorphisms (SNPs). J Biochem Biophys Methods. 2006; 67:87-94
30. Tang J, Li Y, Pan Z, Guo Y, Ma J, Ning S, Xiao P, Lu Z. Single nucleotide variation detection by ligation of universal probes on a 3D poyacrylamide gel DNA microarray. Hum Mutat. 2009; 30:1460-8
31. Choi S Y, Lee K Y, Kim Y E, Bae J W, Oh S K, Kim S Y, Hwang S J, Kim U K, Lee S H. Application of allele-specific primer extension-based microarray for simultaneous multi-gene mutation screening in patients with non-syndromic hearing loss. Int J Mol Med. 2010; 25:315-20
32. Kure S, Shinka T, Sakata Y, Osamu N, Takayanagi M, Tada K, Matsubara Y, Narisawa K. A one-base deletion (183delC) and a missense mutation (D276H) in the T-protein gene from a Japanese family with nonketotic hyperglycinemia. J Hum Genet. 1998; 43:135-7
33. Ko C H, Shen S C, Hsu C S, Chen Y C. Mitochondrial-dependent, reactive oxygen species-independent apoptosis by myricetin: roles of protein kinase C, cytochrome c, and caspase cascade. Biochem Pharmacol. 2005; 69:913-27
34. Alderborn A, Kristofferson A, Hammerling U. Determination of single-nucleotide polymorphisms by real-time pyrophosphate DNA sequencing. Genome Res. 2000; 10:1249-58.
35. Chen D C, Saarela J, Nuotio I et al. Comparison of GenFlex tag array and pyrosequencing in SNP genotyping. J Mol Diagn. 2003; 5:243-9.
36. Sinclair A, Arnold C, Woodford N. Rapid detection and estimation by pyrosequencing of 23S rRNA genes with a single nucleotide polymorphism conferring linezolid resistance in Enterococci. Antimicrob Agents Chemother. 2003; 47:3620-2.
37. Adelson M E, Feola M, Trama J, Tilton R C, Mordechai E. Simultaneous detection of herpes simplex virus types 1 and 2 by real-time PCR and pyrosequencing. J Clin Virol. 2005; 33:25-34.
38. Galbiati S, Foglieni B, Travi M et al. Peptide-nucleic acid-mediated enriched polymerase chain reaction as a key point for non-invasive prenatal diagnosis of beta-thalassemia. Haematologica. 2008; 93:610-4.

All publications, patents, patent applications, accession numbers for nucleic acid or amino acid sequences cited herein are hereby incorporated by reference in their entirety for all purposes to the same extent as if each individual publication, patent, patent application, nucleic acid or amino acid sequence were specifically and individually indicated to be so incorporated by reference.

1. A method for risk assessment, diagnosis, subdiagnosis or prognosis of atherosclerosis, coronary heart disease (CHD) or an atherosclerosis or CHD related condition in a mammalian subject comprising:
a) providing a biological sample selected from the group consisting of a blood, saliva, urine, mucosal, and hair shaft sample taken from the subject;
b) detecting one or more CHD and/or atherosclerosis or related phenotype associated biomarkers in said sample, wherein the biomarkers are related to one or more genes selected from the group consisting of MT-ND5, MT-RNR1, MT-TL1, MT-TL2, MT-ND1, MT-ND6 and MT-CYB genes, which encode subunit 5 of NADH dehydrogenase, 12S rRNA, tRNA-Leu 1, tRNA-Leu 2, subunits 1 and 6 of NADH dehydrogenase, and cytochrome B, respectively, or said biomarkers are related to one or more polypeptides encoded by said genes, and;
c) comparing the biomarker data from the subject to biomarker data from healthy and diseased people to make risk assessment, diagnosis, subdiagnosis or prognosis of atherosclerosis, CHD or a CHD related condition.

2. The method according to claim 1, wherein said atherosclerosis or CHD related condition comprises coronary heart disease, such as myocardial infarction and angina pectoris, and cerebrovascular disease, congestive heart failure, claudication or other clinical manifestation of atherosclerosis or arteriosclerosis, hypertension, obesity or type 2 diabetes mellitus.

3. The method according to claim 1, wherein at least one biomarker is an atherosclerosis, CHD and/or atherosclerosis and/or related phenotype associated polymorphic or heteroplasmic site residing in a genomic region containing a gene selected from the group consisting of MT-ND5, MT-RNR1, MT-TL1, MT-TL2, MT-ND1, MT-ND6 and MT-CYB, which encode subunit 5 of NADH dehydrogenase, 12S rRNA, tRNA-Leu 1, tRNA-Leu 2, subunits 1 and 6 of NADH dehydrogenase, and cytochrome B, respectively.

4. The method according to claim 1, wherein at least one biomarker is selected from the heteroplasmy markers set forth in Table 3.

5. The method according to claim 1, wherein the biomarker is the level of G→A heteroplasmy of the locus 13513 in mtDNA.

6. The method according to claim 1, wherein the biomarker is the level of C→T heteroplasmy of the locus 3256 in mtDNA.

7. The method according to claim 1, wherein the biomarker is the level of G→A heteroplasmy of the locus 12315 in mtDNA.

8. The method according to claim 1, wherein the biomarker is the level of G→A heteroplasmy of the locus 15059 in mtDNA.

9. The method according to claim 1, wherein at least one biomarker is an expression product of a gene selected from the group consisting of MT-ND5, MT-RNR1, MT-TL1, MT-TL2, MT-ND1, MT-ND6 and MT-CYB, which encode subunit 5 of NADH dehydrogenase, 12S rRNA, tRNA-Leu 1, tRNA-Leu 2, subunits 1 and 6 of NADH dehydrogenase, and cytochrome B, respectively.

10. The method according to claim 1 further comprising a step of combining non-genetic information with the biomarker data to make risk assessment, diagnosis or prognosis of atherosclerosis, CHD or a CHD related condition for a subject.

11. The method according to claim 10, wherein the non-genetic information comprises age, gender, ethnicity, socioeconomic status, history of manifestations of atherosclerosis, other medical history of the subject, family history of relevant conditions, psychological traits and states, behaviour patterns and habits, biochemical measurements and clinical measurements.

12. The method according to claim 1 further comprising a step of calculating the risk of atherosclerosis, CHD or a CHD related condition using a logistic regression equation as follows:
Risk of CHD=[1+e^{-(a+Σ(bi*Xi)}]⁻¹, where e is the base of the natural logarithm, Xᵢ are variables associated with the risk of CHD, bᵢ are coefficients of these variables in the logistic function, and a is the constant term in the logistic function.

13. The method according to any one of the preceding claim for risk assessment, diagnosis, subdiagnosis or prognosis of atherosclerosis, coronary heart disease (CHD) or an atherosclerosis or CHD related condition in a human subject comprising:
a) providing a biological sample selected from the group consisting of a blood, saliva, urine, mucosal, and hair shaft sample taken from the subject;
b) detecting the level of heteroplasmy of one or more CHD and/or atherosclerosis associated mtDNA biomarkers in said sample, wherein the biomarkers are selected from the group consisting of 13513 G→A of the gene encoding subunit 5 of NADH dehydrogenase, 652 ins/del G and 1555 A→G of 12S rRNA gene, 3256 C→T of tRNA-Leu 1 gene, 3336 T→C of the gene encoding subunit 1 of NADH dehydrogenase, 12315 G→A of tRNA-Leu 2 gene, 14459 G→A of the gene encoding subunit 6 of NADH dehydrogenase, and 14846 G→A and 15059 G→A of cytochrome B gene;
c) comparing the biomarker data from the subject to biomarker data from healthy and diseased people to make risk assessment, diagnosis, subdiagnosis or prognosis of atherosclerosis, CHD or a CHD related condition.

14. A test kit for risk assessment, diagnosis, subdiagnosis or prognosis of atherosclerosis, CHD or a CHD related condition comprising:
a) reagents, materials and protocols for assessing type and/or level of one or more CHD and/or atherosclerosis phenotype associated biomarkers in a biological sample selected from the group consisting of a blood, saliva, urine, mucosal, and hair shaft sample, wherein the biomarkers are related to one or more genes selected from the group consisting of MT-ND5, MT-RNR1, MT-TL1, MT-TL2, MT-ND1, MT-ND6 and MT-CYB, which encode subunit 5 of NADH dehydrogenase, 12S rRNA, tRNA-Leu 1, tRNA-Leu 2, subunits 1 and 6 of NADH dehydrogenase, and cytochrome B, respectively, or said biomarkers are related to one or more polypeptides encoded by said genes, and;
b) instructions, manual and software for comparing the biomarker data from a subject to biomarker data from healthy and diseased people to make risk assessment, diagnosis, subdiagnosis or prognosis of atherosclerosis, CHD or a CHD related condition.

15. The test kit according to claim 14, wherein at least one biomarker is a CHD and/or atherosclerosis associated polymorphic site residing in a genomic region containing a gene selected from the group consisting of MT-ND5, MT-RNR1, MT-TL1, MT-TL2, MT-ND1, MT-ND6 and MT-CYB, which encode subunit 5 of NADH dehydrogenase, 12S rRNA, tRNA-Leu 1, tRNA-Leu 2, subunits 1 and 6 of NADH dehydrogenase, and cytochrome B, respectively.

16. The test kit according to claim 14, wherein at least one biomarker is selected from the heteroplasmy markers set forth in Table 3.

17. The test kit according to claim 14, wherein at least one biomarker is a polymorphic site associated with one or more of the heteroplasmy markers set forth in Table 3.

18. The test kit according to claim 14, wherein at least one biomarker is an expression product of a gene selected from the group consisting of MT-ND5, MT-RNR1, MT-TL1, MT-TL2, MT-ND1, MT-ND6 and MT-CYB, which encode subunit 5 of NADH dehydrogenase, 12S rRNA, tRNA-Leu 1, tRNA-Leu 2, subunits 1 and 6 of NADH dehydrogenase, and cytochrome B, respectively.

19. The test kit according to claim 14, wherein said test kit is for selecting efficient and safe therapy to prevent or treat atherosclerosis, CHD or a CHD related condition in a subject having increased risk of atherosclerosis, CHD or a CHD related condition.

20. The test kit according to claim 14 wherein said test kit is for predicting efficiency or monitoring the effect of a therapy used to prevent or treat atherosclerosis, CHD or a CHD related condition in a subject having increased risk of atherosclerosis, CHD or a CHD related condition.

21. The test kit according to claim 14, wherein said test kit is for diagnosing a subtype of CHD in a subject having atherosclerosis, CHD or a CHD related condition.

22. The test kit according to claim 14 further comprising a questionnaire and instructions for collecting personal and clinical information from the subject, and software and instructions for combining personal and clinical information with biomarker data to make risk assessment, diagnosis, subdiagnosis or prognosis of atherosclerosis, CHD or a CHD related condition.

23. The test kit according to claim 14 further comprising a step of calculating the risk of atherosclerosis, CHD or a CHD related condition using a logistic regression equation as follows:
Risk of CHD=[1+e^{-(a+Σ(bi*Xi)}]⁻¹, where e is the base of the natural logarithm, Xᵢ are variables associated with the risk of CHD, bᵢ are coefficients of these variables in the logistic function, and a is the constant term in the logistic function.

24. The test kit according to claim 14 comprising a PCR primer set for amplifying at least one of said biomarkers.

25. The test kit according to claim 14 comprising a capturing nucleic acid probe set specifically binding to at least one of said biomarkers.

26. The test kit according to claim 14 comprising a microarray or multiwell plate to assess said biomarkers.

27. The test kit according to claim 14, wherein the biomarkers are selected from the group consisting of 13513 G→A of the gene encoding subunit 5 of NADH dehydrogenase, 652 ins/del G and 1555 A→G of 12S rRNA gene, 3256 C→T of tRNA-Leu gene, 3336 T→C of the gene encoding subunit 1 of NADH dehydrogenase, 12315 G→A of tRNA-Leu gene, 14459 G→A of the gene encoding subunit 6 of NADH dehydrogenase, and 14846 G→A and 15059 G→A of cytochrome B gene

28. A method for screening agents for preventing or treating atherosclerosis, CHD or a CHD related condition in a mammal comprising determining the effect of an agent either on a metabolic pathway related to a polypeptide or a RNA molecule encoded by a CHD and/or atherosclerosis associated gene selected from the group consisting of MT-ND5, MT-RNR1, MT-TL1, MT-TL2, MT-ND1, MT-ND6 and MT-CYB, which encode subunit 5 of NADH dehydrogenase, 12S rRNA, tRNA-Leu 1, tRNA-Leu 2, subunits 1 and 6 of NADH dehydrogenase, and cytochrome B, respectively, in living cells; wherein an agent altering activity of a metabolic pathway is considered useful in prevention or treatment of atherosclerosis, CHD or a CHD related condition.

29. The method according to claim 28, wherein said agent is administered to a model system or organism, and wherein an agent altering or modulating expression, biological activity or function of a CHD, atherosclerosis, hypertension, obesity and/or type 2 diabetes associated gene selected from the group consisting of MT-ND5, MT-RNR1, MT-TL1, MT-TL2, MT-ND1, MT-ND6 and MT-CYB, which encode subunit 5 of NADH dehydrogenase, 12S rRNA, tRNA-Leu 1, tRNA-Leu 2, subunits 1 and 6 of NADH dehydrogenase, and cytochrome B, respectively, or it's encoded polypeptide is considered useful in prevention or treatment of atherosclerosis, CHD or a CHD related condition.

30. The method according to claim 28, wherein the model system or organism comprises cultured microbial, insect or mammalian cells, mammalian tissues, organs or organ systems or non-human transgenic animals expressing a CHD, atherosclerosis, hypertension and/or obesity and type 2 diabetes associated gene selected from the group consisting of MT-ND5, MT-RNR1, MT-TL1, MT-TL2, MT-ND1, MT-ND6 and MT-CYB, which encode subunit 5 of NADH dehydrogenase, 12S rRNA, tRNA-Leu 1, tRNA-Leu 2, subunits 1 and 6 of NADH dehydrogenase, and cytochrome B, respectively.

31. Recombinant MT-ND5, MT-RNR1, MT-TL1, MT-TL2, MT-ND1, MT-ND6 and MT-CYB, which encode 12S rRNA, tRNA-Leu, cytochrome B, and subunits 1, 5, and 6 NADH dehydrogenase or analogs of MT-RNR1, MT-TL1, MT-TL2, MT-ND1, MT-ND2, MT-ND5, MT-ND6 and MT-CYB, which encode subunit 5 of NADH dehydrogenase, 12S rRNA, tRNA-Leu 1, tRNA-Leu 2, subunits 1 and 6 of NADH dehydrogenase, and cytochrome B, respectively, for use in the treatment of atherosclerosis, CHD or a CHD related condition.

32. Method for treatment of atherosclerosis, CHD or a CHD related condition, wherein a pharmaceutically effective amount of antibodies, miRNA, siRNA or other form of RNA interference agent of MT-ND5, MT-RNR1, MT-TL1, MT-TL2, MT-ND1, MT-ND6 and MT-CYB, or pharmaceutically effective amount of recombinant, analogs of subunit 5 of NADH dehydrogenase, 12S rRNA, tRNA-Leu 1, tRNA-Leu 2, subunits 1 and 6 of NADH dehydrogenase, and cytochrome B is administered to a patient in need of such treatment.

33. Method for gene therapy of atherosclerosis, CHD or a CHD related condition, wherein a pharmaceutically effective amount of a vector is administered to transfect 12S rRNA, tRNA-Leu, cytochrome B, and subunits 1 and 6 of NADH dehydrogenase to a patient in need of such treatment.

34. Method for treatment of atherosclerosis, CHD or a CHD related condition, wherein a pharmaceutically effective amount of recombinant or analogs of NADH dehydrogenase, mutated G→A at the locus 13513 of subunit 5 or recombinant or analog of the subunit 5 of NADH dehydrogenase, mutated G→A at the locus 13513 is administered to a patient in need of such treatment.

35. Method for gene therapy of atherosclerosis, CHD or a CHD related condition, wherein a vector is used to insert effective amount of NADH dehydrogenase, mutated G→A at the locus 13513 of subunit 5 to administer to a patient in need of such treatment.

36. Method for treatment of atherosclerosis, CHD or a CHD related condition, wherein a pharmaceutically effective amount of siRNA or other gene silencing agent or other inhibitor of RNA or antibody or inhibitor of the protein of mutated 652 del G and 1555 A→G of 12S rRNA gene, 3256 C→T of tRNA-Leu gene, 3336 T→C of the gene encoding subunit 1 of NADH dehydrogenase, 12315 G→A of tRNA-Leu gene, 14459 G→A of the gene encoding subunit 6 of NADH dehydrogenase, and 14846 G→A and 15059 G→A of cytochrome B gene is administered to a patient in need of such treatment.
